SEQUENCE LISTING (20 sequences)

SEQ ID NO: 1 - protein, 99 aa, Artificial Sequence
  Description of Artificial Sequence Synthetic polypeptide
RRVGDELEKEYERAFSSFSAQLHVTPTTARELFGQVATQLFSDGNINWGRVVALFSFGGFLALKLVDKELEDLVSRLASFLSEFLAKTLANWLRENGGW

SEQ ID NO: 2 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - protein, 25 aa, Homo sapiens
TGYDNREIVMKYIHYKLSQRGYEWD

SEQ ID NO: 4 - protein, 175 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 5 - protein, 30 aa, Homo sapiens
FGYIYRLAQDYLQCVLQIPQPGSGPSKTSR

SEQ ID NO: 6 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 7 - protein, 27 aa, Homo sapiens
MSQSNRELVVDFLSYKLSQKGYSWSQF

SEQ ID NO: 8 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 9 - protein, 24 aa, Homo sapiens
SAPDTRALVADFVGYKLRQKGYVC

SEQ ID NO: 10 - protein, 350 aa, Homo sapiens [sequence omitted]

SEQ ID NO: 11 - protein, 20 aa, Homo sapiens
DLYRQSLEIISRYLREQATG

SEQ ID NO: 12 - protein, 24 aa, Artificial Sequence
  Description of Artificial Sequence Synthetic peptide
PRLDIRGLVVDYVTYKLSQNGYEW

SEQ ID NO: 13 - DNA, 720 bp, Homo sapiens
  CDS (1)..(717)
atggcgcacgctgggagaagtggttacgataaccgggagatagtgatgaagtacatccattataagctgtcgcagaggggctacgagtgggatgcgggagatgtgggcgccgcgcccccgggggccgcccccgcaccgggcttcttctcctcccagcccgggcacacgccccatccagccgcatcccgggacccggtcgccaggacctcgccactacagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggatgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 14 - protein, 239 aa, Homo sapiens [sequence omitted]

SEQ ID NO: 15 - protein, 222 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFHHHHHHHHHH

SEQ ID NO: 16 - protein, 158 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSHHHHHH

SEQ ID NO: 17 - protein, 25 aa, Artificial Sequence
  Description of Artificial Sequence Synthetic peptide
TGYDNREIVMKYIHYKLSQRGYEWD

SEQ ID NO: 18 - protein, 25 aa, Artificial Sequence
  Description of Artificial Sequence Synthetic peptide
FGYIYRLAQDYLQCVLQIPZPGSGP

SEQ ID NO: 19 - protein, 27 aa, Artificial Sequence
  Description of Artificial Sequence Synthetic peptide
MSQSNRELVVDFLSYKLSQKGYSWSQF

SEQ ID NO: 20 - protein, 25 aa, Artificial Sequence
  Description of Artificial Sequence Synthetic peptide
TWHMYGNQRDYIGDRSKIVYKLEYE

(19) United States
(12) Patent Application Publication
(10) Pub. No.: US 2011/0021433 A1
(43) Pub. Date: Jan. 27, 2011

(54) METHODS FOR TREATING OR PREVENTING HEART FAILURE

(76) Inventors: John M. Harlan, Seattle, WA (US); Robert K. Winn, Seattle, WA (US); Akiko Iwata, Seattle, WA (US)

Correspondence Address: FENWICK & WEST LLP, SILICON VALLEY CENTER, 801 CALIFORNIA STREET, MOUNTAIN VIEW, CA, 94041, US

(21) Appl. No.: 12/697,978

(22) Filed: Feb. 1, 2010

Related U.S. Application Data
(63) Continuation-in-part of application No. 11/576,591, filed on Jan. 18, 2008.
(60) Provisional application No. 61/148,768, filed on Jan. 30, 2009.
(60) Provisional application No. 60/714,511, filed on Oct. 4, 2004.
(60) Provisional application No. 60/709,053, filed on Aug. 16, 2005.

(51) Int. Cl. A61K 38/16; A61K 38/10; A61P 9/00

(52) U.S. Cl. 514/16.4

(57) ABSTRACT

The invention provides methods for inhibiting cell death or inflammation in a mammal brought about by diseases or conditions such as heart failure, wherein the method comprises administering to a mammal in need thereof a Bcl protein in an amount sufficient to inhibit cell death and inflammation in the mammal.

### CROSS REFERENCE TO RELATED APPLICATIONS

This application is a continuation-in-part of U.S. application Ser. No. 11/576,591 filed Jan. 18, 2008 and titled “Methods Of Inhibiting Cell Death Or Inflammation In A Mammal,” which is a U.S. 35 USC §371 of international Application No. PCT/US2005/035666 filed Oct. 4, 2005, which claims priority to U.S. Provisional Application No. 60/714,511 filed Oct. 4, 2004 and U.S. Provisional Application No. 60/709,053 filed Aug. 16, 2005, and this application also claims the benefit of U.S. Provisional Application No. 61/148,768 filed Jan. 30, 2009 which are incorporated by reference in their entirety.

This application is related to U.S. patent application Ser. No. 12/514,436 filed and titled “Methods Of Inhibiting Cell Death Or Inflammation In A Mammal”, which is a U.S. 35 USC §371 of international Application No. PCT/US2007/084052, filed Nov. 8, 2007 which is the nonprovisional of U.S. Provisional Application No. 60/857,913, filed Nov. 10, 2006. This application is also related to U.S. Provisional Application No. 61/148,754 filed Jan. 30, 2009, the contents of which are all hereby incorporated by reference.

### STATEMENT REGARDING FEDERALLY SPONSORED RESEARCH OR DEVELOPMENT

This invention was made with government support under Grant Nos. GM66197, GM42686 and HL72262 awarded by the National Institutes of Health. The government has certain rights in the invention.

### FIELD

This invention relates to methods for preventing or treating heart failure, including chronic heart failure, chronic heart failure at a phase of acute exacerbation and heart failure at a phase of transition to chronic heart failure.

### BACKGROUND

Diseases of the cardiovascular system are a leading worldwide cause of mortality and morbidity. Heart failure has been increasing in prevalence. Heart failure is characterized by an inability of the heart to deliver sufficient blood to the various organs of the body. Current estimates indicate that over 5 million Americans carry the diagnosis of heart failure with nearly 500,000 new cases diagnosed each year and 250,000 deaths per year attributed to this disease. Despite significant therapeutic accomplishments in the past two decades, heart failure continues to increase in incidence reaching epidemic proportions and presenting a major economic burden in developed countries.

Acute heart failure is defined as a condition of cardiac dysfunction caused by rapid deterioration of the circulation dynamics. Common symptoms include marked dyspnea due to pulmonary congestion or cardiogenic shock due to low cardiac output, expectoration of foamy sputum, oliguria or anuria, cold extremities, lowering of blood pressure, cold sweat and tachycardia (sometimes bradycardia). Unless treated immediately, patients suffering from acute heart failure will soon die.

On the other hand, chronic heart failure is an equilibrium state in which compensation mechanisms such as cardiac hypertrophy work because of slow progress of heart failure observed in old myocardial infarction and dilated cardiomyopathy; common symptoms include shortness of breath, fatigability, reduced exercise tolerance, enlargement of the liver and spleen, edema and varicosis of peripheral veins. Therefore, improving the prognosis of life is the ultimate goal of treatment of chronic heart failure and the goals of management are to improve exercise tolerance and the quality of life (QOL).

Thus, acute and chronic heart failures differ in pathological conditions and the object of treatment and it is important to choose the right therapeutic drugs specific to each type of heart failure. For patients with acute heart failure, saving their life is an ultimate goal, cardiotonic preparations are chosen as therapeutics that enhance the reduced myocardial contractility and which can improve the aggravated circulation dynamics. However, if cardiotonic preparations are administered to patients with chronic heart failure who are in stable condition, the already fatigued heart muscle is contracted more by added stimulation with the cardiotonic agent and the condition progresses adversely.

Since most heart failures can not be treated by causal therapy, the symptoms change gradually for the worse with the lapse of time. Even though the cardiac function is temporarily recovered by a temporary symptomatic therapy, the function changes for the worse later and it is impossible to affect a permanent cure. The worsening rate varies depending on various conditions such as the kinds of underline diseases, severity of the disease, effectiveness of therapy, and living environment. Besides, the patients sometimes unexpectedly suddenly die, even while they have been in the favorable course of recovering.

Accordingly, there is a continuing need for methods and compositions for treating or preventing heart failure.

### SUMMARY

Heart failure can be defined as a complex clinical syndrome characterized by abnormalities of left ventricular function and neurohormonal regulation, which in turn can result in effort intolerance, fluid retention, and reduced longevity.

The heart failure is classified to “acute heart failure” and “chronic heart failure” or “chronic heart failure in acute exacerbation” based on the clinical symptoms and the disease course.

The therapeutic measures for heart failure are entirely different depending on the conditions, i.e., whether it is in the chronic phase or in the acute phase. For the treatment of the chronic heart failure, it is treated so as to release the remaining heart failure symptom and to maintain in the stable state so that the symptom falls into acute exacerbation. On the other hand, when the patient falls in acute exacerbation, the symptom can possibly change rapidly for the worse and there is threat to life, and hence, from this viewpoint, it is necessary to take a life-saving measure by temporarily controlling the breath and blood pressure and administering a cardiotonic drug so as to moderate and stabilize the clinical symptoms, in hemodynamic viewpoint, by increasing cardiac output, decreasing intravascular circulation volume, and increasing renal blood flow.

Programmed cell death is a normal and necessary part of mammalian development. For example, the development of separate fingers in a human fetus requires the programmed cell death of tissue between the developing fingers. The biochemical processes that cause programmed cell death can be triggered, however, by a variety of diseases and injuries. For example, programmed cell death can be triggered by traumatic injury, stroke, myocardial infarction, organ transplantation, and mesenteric and peripheral vascular disease. Cell death and inflammation is also brought about by chronic heart failure, chronic heart failure at a phase of acute exacerbation, a phase of transition to chronic heart failure or the like. The programmed cell death further undermines the health of the injured or diseased organism.

Each of the foregoing types of diseases and injuries typically include some ischemia and reperfusion injury, which occurs when previously interrupted blood flow is restored to living tissue. For example, blockage of a coronary artery can cause cardiac muscle death due to the temporary lack of blood supply to the cardiac tissue. Additional muscle can die when blood flow is restored to the cardiac muscle by the administration of thrombolytic drugs.

Chronic and acute inflammation can also damage or kill living cells in a mammal For example, the inflammation associated with emphysema causes lung damage over time. Inflammation can trigger programmed cell death, or can damage living tissue by some other mechanism. Accordingly, there is a continuing need for methods and compositions for inhibiting cell death and inflammation in a mammal

The invention provides methods for inhibiting cell death or inflammation in a mammal comprising administering to a mammal in need thereof a Bcl protein in an amount sufficient to inhibit cell death and inflammation in the mammal In some aspects, cell death is inhibited in the mammal In other aspects, the inflammation is inhibited in the mammal In some such aspects, the cell death and/or inflammation caused by heart failure is inhibited. In some aspects the heart failure is acute heart failure, chronic heart failure, chronic heart failure at a phase of acute exacerbation, or a phase of transition to chronic heart failure. In some aspects, the mammal is a human. In other aspects, the administering follows an acute administration regime.

In certain aspects, the Bcl protein is selected from the group consisting of: (a) protein comprising an amino acid sequence that is at least 35% identical to the amino acid sequence set forth in SEQ ID NO:1; (b) a protein comprising at least 12 amino acids, wherein the protein is at least 50% similar to a segment of a Bcl-2 protein, wherein the Bcl-2 protein consists of the amino acid sequence set forth in SEQ ID NO:2; (c) a protein comprising at least 12 amino acids, wherein the protein is at least 50% similar to a segment of an A-1 protein, wherein the A-1 protein consists of the amino acid sequence set forth in SEQ ID NO:4; (d) a protein comprising at least 12 amino acids, wherein the protein is at least 50% similar to a segment of a Bcl-X protein, wherein the Bcl-X protein consists of the amino acid sequence set forth in SEQ ID NO:6; (e) a protein comprising at least 12 amino acids, wherein the protein is at least 50% similar to a segment of a Bcl-W protein, wherein the Bcl-W protein consists of the amino acid sequence set forth in SEQ ID NO:8; (f) a protein comprising at least 12 amino acids, wherein the protein is at least 50% similar to a segment of an Mcl-1 protein, wherein the Mcl-1 protein consists of the amino acid sequence set forth in SEQ ID NO:10; and (g) a protein that is at least 50% similar to a BH4 domain consisting of the amino acid sequence set forth in SEQ ID NO:12.

In other aspects, the administering follows a chronic administration regime. In some aspects, the Bcl protein is administered intravenously, orally, or transdermally. In some aspects, the Bcl protein is administered prophylactically to a mammalian subject. In some such aspects, the mammalian subject is suffering heart failure. In other aspects, the Bcl protein is administered to the mammalian subject in an amount from 0.5 μg/kg/day to 50 μg/kg/day for a period of time sufficient to inhibit a member of the group consisting of cell death and inflammation in the mammal In some aspects, the Bcl protein is administered to the mammalian subject for a period of from 1 day to 20 days. In other aspects, the Bcl protein is administered to the mammalian subject on multiple occasions. In some aspects, the Bcl protein is administered to the mammalian subject daily.

### BRIEF DESCRIPTION OF DRAWINGS

The foregoing aspects and many of the attendant advantages of this invention will become more readily appreciated as the same become better understood by reference to the following detailed description, when taken in conjunction with the accompanying drawings, wherein.

FIG. 1 shows a bar chart of creatine kinase concentration in blood plasma of eleven transgenic mice that expressed exogenous human Bcl-2 (hBcl-2) in their myeloid cells (identified as Bcl-2 in FIG. 1), and a bar chart of creatine kinase concentration in blood plasma of nine non-transgenic, control, C57BL/6 mice that did not express exogenous hBcl-2 in their myeloid cells. Creatine kinase concentration was measured after the mice had been subjected to ischemia-reperfusion injury as described in Example 1. Creatine kinase concentration is expressed in units per liter (U/L) (*p<0.05).

FIG. 2 shows a bar chart of creatine kinase concentration in blood plasma of eight control C57BL/6 mice (identified as C57 in FIG. 2) that had suffered ischemia-reperfusion injury, and a bar chart of creatine kinase concentration in blood plasma of eight EμT-Bcl-2 mice (identified as EμBcl-2 (T-cell) in FIG. 2) that had suffered ischemic injury. (*p<0.05).

FIG. 3 shows a bar chart of the percentage of TUNEL positive cells in muscle tissue from the legs of six control C57BL/6 mice (abbreviated as C57), five EμT-Bcl-2 mice (abbreviated as EμT) that express hBcl-2 in their T-cells, six EμB-Bcl-2 mice (abbreviated as EμB) that express hBcl-2 in their B-cells, and five hMRP8-myeloid-Bc1-2 mice (abbreviated as hMRP) that express hBcl-2 in their myeloid cells. As described in Example 3, all of the mice had suffered ischemia-reperfusion injury (*p<0.05 versus C57).

FIG. 4 shows a bar chart of creatine kinase concentration, after ischemia and reperfusion, in blood plasma of six mice (identified as tg+ mice) that had received an injection, before ischemia, of blood plasma extracted from mice that express hBcl-2 in their T-lymphocytes; and a bar chart of creatine kinase concentration, after ischemia and reperfusion, in blood plasma of mice (identified as tg−0 mice) that had received an injection, before ischemia, of blood plasma extracted from six littermate control mice that did not express hBc-2 in their T-lymphocytes. (*p<0.05).

FIG. 5 shows a bar chart of creatine kinase concentration in blood plasma of 18 mice that had been injected with Jaws II leukocytes that express hBcl-2 (identified by the abbreviation Bcl-2); a bar chart of creatine kinase concentration in blood plasma of 16 control mice that had been injected with Jaws II leukocytes that express enhanced green fluorescent protein (identified by the abbreviation GFP); and a bar chart of creatine kinase concentration in blood plasma of 12 control mice that had been injected with phosphate buffered saline (identified by the abbreviation PBS). The creatine kinase concentration was measured after the mice had been subjected to ischemic reperfusion injury as described in Example 5 (*p<0.05).

FIG. 6 shows a bar chart of creatine kinase concentration, after ischemia and reperfusion, in blood plasma of ten mice (identified as JAWSII-Bcl-2 in FIG. 6) that had received an injection, before ischemia, of supernatant medium from a culture of hBcl-2-Jaws II cells that express Bcl-2; and a bar chart of creatine kinase concentration, after ischemia and reperfusion, in blood plasma of nine mice (identified as JAWSII-GFP in FIG. 6) that had received an injection, before ischemia, of supernatant medium from a culture of EGFP-Jaws II cells that express enhanced green fluorescent protein. The creatine kinase concentrations were significantly different at p<0.05.

FIG. 7 shows a bar chart of creatine kinase concentration, after ischemia and reperfusion, in blood plasma of 12 mice (identified as rBcl-2) that had received an injection, before ischemia and reperfusion, of recombinant human Bcl-2 (1 μg per mouse); and a bar chart of creatine kinase concentration, after ischemia and reperfusion, in blood plasma of 12 control mice (identified as control) that had received an injection, before ischemia and reperfusion, of either recombinant human ubiquitin or the vehicle solution used for injection of recombinant Bcl-2. There was no difference in the creatine kinase concentration between these two types of controls, and so these control data were combined. The creatine kinase concentrations were significantly different at p<0.05.

FIG. 8 shows a bar chart of the infarct volume (V_{infarct}) as a percentage of the left ventricular volume (V_{LV}) for five hMRP8-Bcl-2 mice that express hBcl-2 in their myeloid cells (identified as Bcl-2/2 mice in FIG. 9) and five C57BL/6 control mice; a bar chart of the infarct volume (V_{infarct}) as a percentage of the area-at-risk volume (V_{AAR}) for C57BL/6 control mice and hMRP8-Bcl-2 mice; and a bar chart of the area-at-risk volume (V_{AAR}) as a percentage of the left ventricular volume (V_{LV}) for C57BL/6 control mice and hMRP8-Bcl-2 mice. V_{infarct}/V_{LV} and V_{infarct}/V_{AAR} was significantly different between Bcl-2/2 mice and C57BL/6 mice at p<0.05.

FIG. 9 shows a bar chart of the infarct volume (V_{infarct}) as a percentage of the left ventricular volume (V_{LV}) for six C57BL/6 control mice and four EμT-Bcl-2 mice (that express Bcl-2 in their T cells); a bar chart of the infarct volume (V_{infarct}) as a percentage of the area-at-risk volume (V_{AAR}) for C57BL/6 control mice and EμT-Bcl-2 mice; and a bar chart of the area-at-risk volume (V_{AAR}) as a percentage of the left ventricular volume (V_{LV}) for C57BL/6 control mice and EμT-Bcl-2 mice. V_{infarct}/V_{LV} and V_{infaret}/V_{AAR} was significantly different between EμT-Bcl-2 mice and C57BL/6 mice at p<0.05

FIG. 10 shows bar charts of the infarct volume (V_{infarct}) as a percentage of the left ventricular volume (V_{LV}), the infarct volume (V_{infarct}) as a percentage of the area-at-risk volume (V_{AAR}), and the area-at-risk volume (V_{AAR}) as a percentage of the left ventricular volume (V_{LV}), for seven C57BL/6 mice that received an injection (before being subjected to myocardial ischemia and reperfusion) of CD11b+ cells that express exogenous human Bcl-2 (these mice are identified as Bcl-2/2 in FIG. 10), and for six C57BL/6 control mice (identified as Littermate Tg−) that had received an infusion (before being subjected to myocardial ischemia and reperfusion) of CD11b+ cells (that did not express exogenous Bcl-2) from their littermates. (*p<0.05)

FIG. 11 shows a survival curve for 12 mice that had been injected with recombinant human Bcl-2 (rBcl-2) prior to cecal ligation and puncture, and 12 control mice that were not injected with rBcl-2 prior to cecal ligation and puncture. The survival curves are significantly different at p<0.05.

### DETAILED DESCRIPTION

The invention provides methods for preventing or treating heart failure, including acute heart failure, chronic heart failure, chronic heart failure at a phase of acute exacerbation or heart failure at a phase of transition to chronic heart failure. The invention further provides administration of a Bcl protein in an amount sufficient to inhibit cell death and inflammation brought about by a variety of diseases and conditions including heart failure (i.e., acute heart failure, chronic heart failure, chronic heart failure at a phase of acute exacerbation or heart failure at a phase of transition to chronic heart failure). Administration of the Bcl protein compositions includes both acute and chronic administration.

Terms used in the claims and specification are defined as set forth below unless otherwise specified.

The term “in situ” refers to processes that occur in a living cell growing separate from a living organism, e.g., growing in tissue culture.

The term “in vivo” refers to processes that occur in a living organism.

The term “heart failure” means a pathological condition characterized by insufficient blood flow from the ventricle to peripheral organs.

The term “acute heart failure” as used herein means a rapid aggravation of circulation dynamics which eventually leads to heart failure.

The term “chronic heart failure at a phase of acute exacerbation” means such a condition that chronic heart failure taking a stable time course deteriorates rapidly due to infection or fatigue caused by overwork, manifesting itself as a similar pathological condition to acute heart failure. The term “chronic heart failure” as used herein means a case of heart failure which progresses so slowly that various compensatory mechanisms work to bring the disease into equilibrium.

The term “a phase of transition to chronic heart failure” means a period after the treatment of acute heart failure during which the state of the disease stabilizes slowly until a transition to chronic heart failure occurs.

“Treating HF” as used herein refers to treating any one or more of the conditions underlying HF, including, without limitation, decreased cardiac contractility, abnormal diastolic compliance, reduced stroke volume, pulmonary congestion, decreased cardiac output, and other diminished hemodynamic functions, while minimizing or attenuating deleterious effects that can be associated with the long-term administration of Bcl proteins such as nausea, diarrhea, severe facial flushing and intermittent tachycardia. “Treating HF” also includes relieving or attenuating symptoms associated with HF.

This invention also provides a method of improving the quality of life in a patient with HF. “Quality of life” refers to one or more of a person's ability to walk, climb stairs, do errands, work around the house, participate in recreational activities, and/or not requiring frequent rest intermittently during activities, and/or the absence of sleeping problems or shortness of breath.

A “patient having HF” refers to a person having Stage B, Stage C, or Stage D heart failure as classified in the American College of Cardiology Guidelines for the Evaluation and Management of Chronic Heart Failure in the Adult. While the American College of Cardiology Guidelines excluded HF in children, for purposes of this invention the methods are to be considered applicable, to any patient, regardless of age.

The methods of the present invention can be practiced on any mammal, such as primates (e.g., human beings), mammals of the genus Canis (e.g., domestic dog), mammals of the genus Felis (e.g., domestic cat), cattle, sheep, horses, goats and pigs.

In the practice of the present invention one or more types of Bcl proteins can be administered to a mammal suffering from cell death (e.g., suffering from a disease that causes cell death, or undergoing a medical treatment that causes cell death, or suffering from an injury that causes cell death). Examples of diseases, or medical treatments, that cause cell death include stroke, myocardial infarction, cardiac arrest, acute coronary syndrome/unstable angina, acute heart failure (AHF), chronic heart failure (CHF), chronic heart failure at a phase of acute exacerbation, cardio-pulmonary by-pass grafting, traumatic shock, organ transplantation, mesenteric, retinal, and peripheral vascular disease, burns, frostbite, re-plantation of limbs and digits, traumatic brain injury, status epilepticus, Parkinson's disease, Huntington's disease, amyotrophic lateral sclerosis, Alzheimer's disease, macular degeneration, acute intracranial hemorrhage, acute renal failure, acute lung injury/adult respiratory distress syndrome, sepsis, meningitis, acute ischemic or alcoholic liver injury, Sjogren's disease, radiation-induced enteritis, and radiation-induced marrow failure.

In the practice of the present invention one or more types of Bcl proteins can be administered to a mammal suffering from inflammation (e.g., suffering from an inflammatory disease, or suffering from an injury that causes inflammation, or undergoing a medical treatment that causes inflammation). Examples of inflammatory diseases include asthma, Crohn's disease, ulcerative colitis, hepatitis (e.g., viral chronic hepatitis), psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, chronic obstructive pulmonary disease, post-influenza pneumonia, SARS, tuberculosis, malaria, sepsis, cerebral malaria, Chagas disease, schistosomiasis, bacteria and viral meningitis, cystic fibrosis, multiple sclerosis, Alzheimer' disease, encephalomyelitis, sickle cell anemia, pancreatitis, transplantation (e.g., host-mediated rejection of transplanted tissue such as hematopoietic stem cells or an organ, graft mediated host response, such as graft vs. host disease), systemic lupus erythematosis, autoimmune diabetes, thyroiditis, and radiation pneumonitis.

Additionally, in the practice of the present invention one or more Bcl proteins can be administered to a mammal that is not suffering from an inflammatory disease or a disease associated with cell death. For example, one or more types of Bcl proteins can be administered prophylactically to a mammal to prevent, or decrease the likelihood of, the onset of cell death or inflammation, or to reduce the severity of cell death and/or inflammation that can subsequently occur. The mammal can be suffering from a disease that can cause cell death and/or inflammation, and the Bcl protein is administered to prevent, or decrease the likelihood of, the onset of cell death or inflammation, or to reduce the severity of cell death and/or inflammation that can subsequently occur. For example, the following categories of human patients can benefit from administration of Bcl to prevent, or decrease the likelihood of, the onset of cell death or inflammation: patients who suffer from transient ischemic attacks at risk for stroke, patients with unstable angina at risk for myocardial infarction, patients with trauma or burns at risk for multiple organ dysfunction, and patients undergoing cardio-pulmonary by-pass grafting at risk for post-operative organ dysfunction.

As used herein, the term “Bcl protein” refers to a protein that inhibits cell death in a mammal when administered to the mammal, and/or inhibits inflammation in a mammal when administered to the mammal, and that is a member of at least one of the following groups of proteins (identified as Groups (a) through (g)).

Group (a): A protein that includes an amino acid sequence that is at least 35% identical (e.g., at least 40%, or at least 45%, or at least 50%, or at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical)) to the amino acid sequence set forth in SEQ ID NO:1:

(SEQ ID NO: 1)
RRVGDELEKEYERAFSSFSAQLHVTPTTARELFGQVATQLFSDGNINWGR
VVALFSFGGFLALKLVDKELEDLVSRLASFLSEFLAKTLANWLRENGGW.

The amino acid sequence set forth in SEQ ID NO:1 is a consensus sequence for the Bcl domain for members of the Bcl-2 family of proteins.

Group (b): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of the Bcl-2 protein consisting of the amino acid sequence set forth in SEQ ID NO:2 (GenBank accession number AAH27258). In some aspects, the protein is at least 50% similar to the following segment of Bcl-2 protein: TGYDNREIVMKYIHYKLSQRGYEWD (SEQ ID NO:3). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-2 protein consisting of the amino acid sequence set forth in SEQ ID NO:2. The Bcl-2 class of proteins are intracellular cytoplasmic proteins that inhibit cell death (see, e.g., J. M. Adams and S. Cory, Science 281:1322-1326 (Aug. 28, 1998); S. Cory, et al., Oncogene 22:8590-8607, 2003).

Group (c): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of an A-1 protein (also referred to as a Bfl-1 protein), wherein the A-1 protein consists of the amino acid sequence set forth in SEQ ID NO:4 (GenBank accession number AAC50438). In some aspects, the protein is at least 50% similar to the following segment of A-1 protein: FGYIYRLAQDYLQCVLQIPQPGSGPSKTSR (SEQ ID NO:5). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the A-1 protein consisting of the amino acid sequence set forth in SEQ ID NO:4. A-1 proteins are homologs of Bcl-2 and are intracellular cytoplasmic proteins that inhibit apoptosis (see, e.g., A. Karsan, et al., Blood 87(8):3089-3096, Apr. 15, 1996; S. S. Choi et al., Mammalian Genome 8:781-782, 1997).

Group (d): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of a Bcl-X protein, wherein the Bcl-X protein consists of the amino acid sequence set forth in SEQ ID NO:6 (GenBank accession number Q07817). In some aspects, the protein is at least 50% similar to the following segment of Bcl-X protein: MSQSNRELVVDFLSYKLSQKGYSWSQF (SEQ ID NO:7). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-X protein consisting of the amino acid sequence set forth in SEQ ID NO:6. Bcl-X proteins are homologs of Bcl-2 and are intracellular cytoplasmic proteins that inhibit apoptosis (see, e.g., L. H. Boise, et al., Cell 74(4):597-608, Aug. 27, 1993.

Group (e): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of a Bcl-W protein consisting of the amino acid sequence set forth in SEQ ID NO:8 (GenBank accession number AAB09055). In some aspects, the protein is at least 50% similar to the following segment of Bcl-W protein: SAPDTRALVADFVGYKLRQKGYVC (SEQ ID NO:9). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-W protein consisting of the amino acid sequence set forth in SEQ ID NO:8. Bcl-W proteins are homologs of Bcl-2, and are intracellular cytoplasmic proteins that inhibit apoptosis (see, e.g., L. Gibson, et al., Oncogene 13(4):665-675, Aug. 15, 1996).

Group (f): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of an Mcl-1 protein, wherein the Mcl-1 protein consists of the amino acid sequence set forth in SEQ ID NO:10 (GenBank accession number AAF64255). In some aspects, the protein is at least 50% similar to the following segment of Mcl-1 protein: DLYRQSLEIISRYLREQATG (SEQ ID NO:11). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Mcl-1 protein consisting of the amino acid sequence set forth in SEQ ID NO:10. Mcl-1 proteins are homologs of Bcl-2 and are intracellular cytoplasmic proteins that inhibit apoptosis.

Group (g): A protein that is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a BH4 domain consisting of the following amino acid sequence: PRLDIRGLVVDYVTYKLSQNGYEW (SEQ ID NO:12). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to the BH4 domain consisting of the amino acid sequence set forth in SEQ ID NO:12. BH4 (Bcl-2 homology domain 4) is an N-terminal domain found in Bcl-2, Bcl-X, and Bcl-W proteins. The amino acid sequence set forth in SEQ ID NO:12 is a consensus sequence for the BH4 domain of the Bcl-2 family of proteins.

As used herein, the term “protein” includes proteins having at least 12 amino acids.

As used herein in connection with proteins useful in the practice of the present invention, the term “segment” refers to at least 12 contiguous amino acids, and can include the complete amino acid sequence of a protein.

As used herein, “Bcl protein”, “BCL2”, and “Bcl-2” are used interchangeably unless otherwise noted.

Representative examples of amino acid sequences of Bcl-2 proteins, useful in the practice of the present invention, are set forth in the protein database accessible through the Entrez search tool of the National Center for Biotechnology Information, U.S. National Library of Medicine, 8600 Rockville Pike, Bethesda, Md. 20894, under the following accession numbers (the amino acid sequences of each of the identified Bcl-2 proteins are incorporated herein by reference): AAN17784.1; AAB17352.1; AAC53460.1; AAK15454.1; AAC53458.1; AAB17354.1; AAA82174.1; AAF88137.1; AAC72232.1; CAC10003.1; AAC53459.1; AAA19257.1; CAA57886.1; AAB17353.1; AAB96881.1; CAA58557.1; AAA82173.1; AAC15799.1; AAA51039.1; AAK15455.1; AAK31308.1; AAK31307.1; CAA80657.1; AAF89532.1; AAK31306.1; BAB85856.2; AAP35872.1; AAH19307.1; BAB71819.1; CAA80061.1; AAF33212.1; AAP36940.1; CAA04597.1; AAB07677.1; AAR92491.1; AAA37281.1; AAH68988.1; AAC60701.2; AAK92201.1; CAB92245.1; AAA37282.1; BAC33767.1; AAP47159.1; AAA77686.1; BAA01978.1; BAC37060.1; AAA77687.1; AAA53662.1; CAA29778.1; AAH27258.1; AA026045.1; AAB53319.1; BAC24136.1; AAA35591.1; CAA78018.1; BAC81344.1; BAD05044.1; AAA51814.1; AAA51813.1; AANO3862.1; CAA57844.1; AAH40369.1; BAB28740.1; BAB62748.1; AAH44130.1; AAH71291.1; AAK81706.1; AAH74505.1; AA064470.1; BAD32203.1; CAA57845.1; AAO64468.1; AAH74021.1; AAC64200.1; AAB86430.1; AAB09056.1; BAB29912.1; BAB23468.1; AAB09055.1; BAA19666.2; AAH73259.1; CAF93123.1; AAO13177.2; CAF96873.1; AAL35559.1; AAP21091.1; AAB97953.1; AAG02475.1; AAK55419.1; AAH27536.1; AAB97956.1; AAH28762.1; AAB97954.1; AAO89009.1; AAP35767.1; AAH16281.1; AAC50438.1; AAC50288.1; CAG46735.1; AAP36152.1; CAG02784.1; CAA70566.1; AAF89533.1; AAO22992.1; AAA03620.1; CAG46760.1; BAC40796.1; CAA73684.1; BAC53619.1; AAH55592.1; AAH66960.1; AAC48806.1; AAF71267.1; AAH04431.1; AAO74828.1; AAA93066.1; AAA74466.1; CAA58997.1; CAG33700.1; BAB85810.1; AAH14175.1; AAA03619.1; AAF98242.1; AAM74949.1; CAD10744.1; AAF71760.1; AAD13295.1; AAH78835.1; AAA75200.1; AAC60700.2; AAH53380.1; AAH18228.1; BAB28776.1; AAA03622.1; AAD31644.1; AAF36411.1; AAC26327.1; AAM34436.1; CAE54428.1; AAH03839.1; AAH21638.1; AAH05427.1; AAC31790.1; BAC77771.1; AAA74467.1; AAF64255.1; AAP36208.1; AAP35286.1; AAH71897.1; AAH17197.1; AAF74821.1; AAD13299.1; AAG00896.1; AAH78871.1; AAH30069.1; AAAC53582.1; AAB87418.1; BAC21258.1; AAF09129.1; AAH63201.1; AAK06406.1; AAR84081.1; AAP36565.1; AAP35936.1; AAH06203.1; AAD51719.1; AAD31645.1; and AAC50142.1.

Bcl proteins include, for example, naturally-occurring Bcl proteins, synthetic Bcl proteins that can incorporate non-natural amino acids, and Bcl fusion proteins in which a protein, peptide, amino acid sequence, or other chemical structure, is attached to a portion (e.g., N-terminal or C-terminal) of a Bcl protein (described in detail below). Representative examples of proteins or chemical structures that can be fused to a Bcl protein include: human serum albumin, an immunoglobulin, polyethylene glycol, or other protein or chemical structure that, for example, increases the serum half-life of the Bcl protein, or increases the efficacy of the Bcl protein, or reduces the immunogenicity of the Bcl protein.

The term “sufficient amount” means an amount sufficient to produce a desired effect, e.g., an amount sufficient to modulate protein aggregation in a cell.

The term “treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

The term “pharmaceutically acceptable” refers to molecular entities and compositions that do not produce adverse, allergic, or other untoward reactions when administered to an animal or a human. Supplementary active ingredients also can be incorporated into the compositions.

“Concomitant administration” of a known therapeutic agent (small molecule, drug, compound, cells, cell line, and the like) with the composition of the present invention means administration of the therapeutic agent (small molecule, drug, compound, cells, cell line, and the like) together with the composition at such time that both the known therapeutic agent (small molecule, drug, compound, cells, cell line, and the like) will have a therapeutic effect or diagnostic effect. Such concomitant administration can involve concurrent (i.e., at the same time), prior, or subsequent administration of the therapeutic agent (small molecule, drug, compound, cells, cell line, and the like) with respect to the administration of a composition of the present invention. A person of ordinary skill in the art would have no difficulty determining the appropriate timing, sequence, and dosages of administration for particular drugs (or other compounds) together with compositions of the present invention.

The term “condition” is used to refer to a disease and/or a response to injury (e.g., trauma, and the like) or treatment (e.g., surgery, transplantation of tissue from a donor, and the like).

The term “ameliorating” refers to any therapeutically beneficial result in the treatment of a disease state, e.g., an ischemic disease state, including prophylaxis, lessening in the severity or progression, remission, or cure thereof.

As used herein, the term “disease” has the meaning generally known and understood in the art and comprises any abnormal condition in the function or well being of a host individual. A diagnosis of a particular disease by a healthcare professional can be made by direct examination and/or consideration of results of one or more diagnostic tests.

The term “inflammatory” when used in reference to a disease, disorder or condition refers to a pathological process caused by, resulting from, or resulting in inflammation that is inappropriate and/or does not resolve in the normal manner. Inflammation results in response to an injury or abnormal stimulation caused by a physical, chemical, or biologic agent; these reactions include the local reactions and resulting morphologic changes, destruction or removal of the injurious material, and responses that lead to repair and healing. Inflammatory disease and conditions can be systemic or localized to particular tissues or organs.

It must be noted that, as used in the specification and the appended claims, the singular forms “a,” “an” and “the” include plural referents unless the context clearly dictates otherwise.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process or method. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a composition” includes a combination of two or more compositions, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

### Peptides

The invention provides composition for treating or preventing a disease or disorder, using a BCL2 peptide of the invention. Exemplary peptides of the invention have an amino acid sequence including those listed herein, and analogs, derivatives, amidated variations and conservative variations thereof, wherein the peptides have activity. The peptides of the invention include the sequences provided herein, as well as the broader groups of peptides having hydrophilic and hydrophobic substitutions, and conservative variations thereof.

“Isolated” when used in reference to a peptide, refers to a peptide substantially free of proteins, lipids, nucleic acids, for example, with which it can be naturally associated. Those of skill in the art can make similar substitutions to achieve peptides with greater biological activity. For example, the invention includes the peptides depicted in sequences provided herein, as well as analogs or derivatives thereof, as long as the bioactivity (e.g., treating or preventing a disease or disorder) of the peptide remains. Minor modifications of the primary amino acid sequence of the peptides of the invention can result in peptides that have substantially equivalent activity as compared to the specific peptides described herein. Such modifications can be deliberate, as by site-directed mutagenesis, or can be spontaneous. All of the peptides produced by these modifications are included herein as long as the biological activity of the original peptide still exists.

Further, deletion of one or more amino acids can also result in a modification of the structure of the resultant molecule without significantly altering its biological activity. This can lead to the development of a smaller active molecule that would also have utility. For example, amino or carboxy terminal amino acids that can not be required for biological activity of the particular peptide can be removed. Peptides of the invention include any analog, homolog, mutant, isomer or derivative of the peptides disclosed in the present invention, so long as the bioactivity as described herein remains. All peptides were synthesized using L amino acids, however, all D forms of the peptides can be synthetically produced. In addition, C-terminal derivatives can be produced, such as C-terminal methyl esters and C-terminal amidates, in order to increase the biological activity of a peptide of the invention. The peptide can be synthesized such that the sequence is reversed whereby the last amino acid in the sequence becomes the first amino acid, and the penultimate amino acid becomes the second amino acid, and so on. It is well known that such reversed peptides usually have similar biologcial activities to the original sequence.

In certain aspects, the peptides of the invention include peptide analogs and peptide mimetics. Indeed, the peptides of the invention include peptides having any of a variety of different modifications, including those described herein.

Peptide analogs of the invention are generally designed and produced by chemical modifications of a lead peptide, including, e.g., any of the particular peptides described herein, such as any of the following sequences disclosed. The present invention further encompasses polypeptides up to about 50 amino acids in length that include the amino acid sequences and functional variants or peptide mimetics of the sequences described herein.

In another aspect, a peptide of the present invention is a pseudopeptide. Pseudopeptides or amide bond surrogates refers to peptides containing chemical modifications of some (or all) of the peptide bonds. The introduction of amide bond surrogates not only decreases peptide degradation but also can significantly modify some of the biochemical properties of the peptides, particularly the conformational flexibility and hydrophobicity.

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers, introduction of cysteines to permit creation of interchain disulphide bonds, or recombinantly though heterologous polypeptides such as Fc regions.

It is known in the art that one or more amino acids can be deleted from the N-terminus or C-terminus without substantial loss of biological function. See, e.g., Ron et al., Biol Chem. 268:2984-2988, 1993. Accordingly, the present invention provides polypeptides having one or more residues deleted from the amino terminus. Similarly, many examples of biologically functional C-terminal deletion mutants are known (see, e.g., Dobeli et al., J. Biotechnology 7:199-216, 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial chaperone polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie et al., Science 247:1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention can be, for example:(i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific aspect of the invention includes polypeptides that include one or more amino acid substitutions that mimic modified amino acids at positions where amino acids that are capable of being modified are normally positioned. Further included are polypeptides where any subset of modifiable amino acids is substituted. For example, a polypeptide that includes three serine residues can be substituted at any one, any two, or all three of said serines. Furthermore, any polypeptide amino acid capable of being modified can be excluded from substitution with a modification-mimicking amino acid.

The present invention is further directed to fragments of the polypeptides of the present invention.

In addition, it should be understood that in certain aspects, the peptides of the present invention include two or more modifications, including, but not limited to those described herein. By taking into the account the features of the peptide drugs on the market or under current development, it is clear that most of the peptides successfully stabilized against proteolysis consist of a mixture of several types of the above described modifications. This conclusion is understood in the light of the knowledge that many different enzymes are implicated in peptide degradation.

### Peptides, Peptide Variants, and Peptide Mimetics

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer Amino acid mimetics refers to chemical compounds that have a structure that is different from the general chemical structure of an amino acid, but which functions in a manner similar to a naturally occurring amino acid. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

“Peptide” as used herein includes peptides that are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acids, or glutamine for asparagine, and the like. Neutral hydrophilic amino acids that can be substituted for one another include asparagine, glutamine, serine and threonine. The term “conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the peptides of the invention. “Cationic” as is used to refer to any peptide that possesses sufficient positively charged amino acids to have a pI (isoelectric point) greater than about 9.0.

The biological activity of the peptides can be determined by standard methods known to those of skill in the art, such as “minimal inhibitory concentration (MIC)” assay described in the present examples, whereby the lowest concentration at which no change in OD is observed for a given period of time is recorded as MIC.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, e.g., a polypeptide fragment of an Bcl protein having Bcl activity as described herein.

Polypeptide mimetic compositions can contain any combination of non-natural structural components, which are typically from three structural groups:a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g.,—C(═O)—CH2— for —C(═O)—NH—), aminomethylene (CH2—NH), ethylene, olefin (CH═CH), ether (CH2—O), thioether (CH2—S), tetrazole (CN4—), thiazole, retroamide, thioamide, or ester (see, e.g., Spatola, 1983, Chemistry and Biochemistry of Amino Acids, Peptides and Proteins 7:267-357).

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl)carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, guanidino-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R— or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a Bcl polypeptide having biological activity of the invention, resulting in modification of the structure of the polypeptide, without significantly altering its biological activity. For example, amino- or carboxyl-terminal, or internal, amino acids that are not required for biologcial activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9:205-223, 1998; Hruby, Curr. Opin. Chem. Biol. 1:114-119, 1997; Ostergaard, Mol. Divers. 3:17-27, 1997; Ostresh, Methods Enzymol. 267:220-234, 1996. Modified peptides of the invention can be further produced by chemical modification methods, see, e.g., Belousov, Nucleic Acids Res. 25:3440-3444, 1997; Frenkel, Free Radic. Biol. Med. 19:373-380, 1995; Blommers, Biochemistry 33:7886-7896, 1994.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, 1995, Therapeutic Peptides and Proteins, Formulation, Processing and Delivery Systems. For example, peptide synthesis can be performed using various solid-phase techniques (see e.g., Roberge, Science 269:202, 1995; Merrifield, Methods Enzymol. 289:3-13, 1997) and automated synthesis can be achieved, e.g., using the ABI 431A Peptide Synthesizer (Perkin Elmer) in accordance with the instructions provided by the manufacturer.

Peptides of the invention can be synthesized by such commonly used methods as t-BOC or FMOC protection of alpha-amino groups. Both methods involve stepwise syntheses whereby a single amino acid is added at each step starting from the C terminus of the peptide (See, Coligan et al., Current Protocols in Immunology, Wiley Interscience, 1991, Unit 9). Peptides of the invention can also be synthesized by the well known solid phase peptide synthesis methods described in Merrifield, J. Am. Chem. Soc. 85:2149, 1962, and Stewart and Young, 1969, Solid Phase Peptides Synthesis 27-62, using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution which is then lyophilized to yield the crude material. This can normally be purified by such techniques as gel filtration on Sephadex G-15 using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Analogs, polypeptide fragment of Bcl protein having biological activity as described herein, are generally designed and produced by chemical modifications of a lead peptide, including, e.g., any of the particular peptides described herein, such as any of the sequences described herein.

The terms “identical” or percent “identity”, in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., 1995 supplement, Current Protocols in Molecular Biology).

Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain aspects, an acceptable range of homology is greater than about 50%. It should be understood that target species can be other than human.

A preferred example of algorithm that is suitable for determining percent sequence identity and sequence similarity are the BLAST and BLAST 2.0 algorithms, which are described in Altschul et al., Nuc. Acids Res. 25:3389-3402, 1977 and Altschul et al., J. Mol. Biol. 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when:the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915, 1989) alignments (B) of 50, expectation (E) of 10, M=5, N=−4, and a comparison of both strands.

### Polypeptides and Functional Variants Thereof

“Polypeptide” includes proteins, fusion proteins, oligopeptides and polypeptide derivatives, with the exception that peptidomimetics are considered to be small molecules herein.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of recombinant protein that has an amino acid sequence that results from the linkage of the amino acid sequences of two or more normally separate polypeptides.

A “protein fragment” is a proteolytic fragment of a larger polypeptide, which can be a protein or a fusion protein. A proteolytic fragment can be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” or “peptide” is a polypeptide having a short amino acid sequence (i.e., 2 to about 200 amino acids). An oligopeptide is generally prepared by chemical synthesis.

Although oligopeptides and protein fragments can be otherwise prepared, it is possible to use recombinant DNA technology and/or in vitro biochemical manipulations. For example, a nucleic acid encoding an amino acid sequence can be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabeled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, can generally be prepared following known techniques. Preferably, synthetic production of the polypeptide of the invention can be according to the solid phase synthetic method. For example, the solid phase synthesis is well understood and is a common method for preparation of polypeptides, as are a variety of modifications of that technique. Merrifield, J. Am. Chem. Soc., 85:2149, 1964; Stewart and Young, 1984, Solid Phase polypeptide Synthesis; Bodansky and Bodanszky, 1984, The Practice of polypeptide Synthesis; Atherton and Sheppard, 1989, Solid Phase polypeptide Synthesis:A Practical Approach.

Alternatively, polypeptides of this invention can be prepared in recombinant systems using polynucleotide sequences encoding the polypeptides.

A “variant” or “functional variant” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention can contain more than one of the following modifications within the same polypeptide. Preferred polypeptide derivatives retain a desirable attribute, which can be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A polypeptide having an amino acid sequence identical to that found in a protein prepared from a natural source is a “wild type” polypeptide. Functional variants of polypeptides can be prepared by chemical synthesis, including without limitation combinatorial synthesis.

Functional variants of polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences can comprise substitutions, deletions and additions of amino acids, with the proviso that such amino acids are naturally occurring amino acids.

Thus, subjecting a nucleic acid that encodes a polypeptide to mutagenesis is one technique that can be used to prepare Functional variants of polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis can be randomly targeted (i.e., mutations can occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids can be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences can be prepared by directed mutagenesis.

As contemplated by this invention, “polypeptide” includes those having one or more chemical modification relative to another polypeptide, i.e., chemically modified polypeptides. The polypeptide from which a chemically modified polypeptide is derived can be a wild type protein, a functional variant protein or a functional variant polypeptide, or polypeptide fragments thereof; an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum. Powell et al., Pharma. Res. 10:1268-1273, 1993. Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. 1993, cited above).

Different host cells will contain different post-translational modification mechanisms that can provide particular types of post-translational modification of a fusion protein if the amino acid sequences required for such modifications is present in the fusion protein. A large number (about 100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and can influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation can vary depending on which host cells are used to produce the fusion protein.

Another type of post-translation modification is the phosphorylation of a free hydroxyl group of the side chain of one or more Ser, Thr or Tyr residues, Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which can have a different chemical version of the methionine residue encoded by a start codon, and these will result in amino termini with different chemical modifications.

For example, many or most bacterial proteins are synthesized with an amino terminal amino acid that is a modified form of methionine, i.e., N-formyl-methionine (fMet). Although the statement is often made that all bacterial proteins are synthesized with an fMet initiator amino acid; although this can be true for E. coli, recent studies have shown that it is not true in the case of other bacteria such as Pseudomonas aeruginosa. Newton et al., J. Biol. Chem. 274:22143-22146, 1999. In any event, in E. coli, the formyl group of fMet is usually enzymatically removed after translation to yield an amino terminal methionine residue, although the entire fMet residue is sometimes removed (see Hershey, 1987, Escherichia coli and Salmonella Typhimurium: Cellular and Molecular Biology, 1: 613-647, and references cited therein.). E. coli mutants that lack the enzymes (such as, e.g., formylase) that catalyze such post-translational modifications will produce proteins having an amino terminal fMet residue (Guillon et al., J. Bacteriol. 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al., Annu. Rev. Physiol. 50:333-344, 1988, and Bradbury et al., Lung Cancer 14:239-251, 1996. About 50% of known endocrine and neuroendocrine peptide hormones are alpha-amidated (Treston et al., Cell Growth Differ 4:911-920, 1993). In most cases, carboxy alpha-amidation is required to activate these peptide hormones.

### Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself For example, polypeptides can exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays 16:683-687, 1994; Cohen and Shatzmiller, J. Mol. Graph. 11:166-173, 1993; Wiley and Rich, Med. Res. Rev. 13:327-384, 1993; Moore, Trends Pharmacol. Sci. 15:124-129, 1994; Hruby, Biopolymers 33:1073-1082, 1993; Bugg et al., Sci. Am. 269:92-98, 1993, all incorporated herein by reference).

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isosteric pseudopeptide bond is a suitable pseudopeptide bond that is known to enhance stability to enzymatic cleavage with no or little loss of biological activity (Couder et al., Int. J. Polypeptide Protein Res. 41:181-184, 1993, incorporated herein by reference). Thus, the amino acid sequences of these compounds can be identical to the sequences of their parent L-amino acid polypeptides, except that one or more of the peptide bonds are replaced by an isosteric pseudopeptide bond. Preferably the most N-terminal peptide bond is substituted, since such a substitution would confer resistance to proteolysis by exopeptidases acting on the N-terminus.

To confer resistance to proteolysis, peptide bonds can also be substituted by retro-inverso pseudopeptide bonds (Dalpozzo et al., Int. J. Polypeptide Protein Res. 41:561-566, incorporated herein by reference). According to this modification, the amino acid sequences of the compounds can be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus

Peptoid derivatives of polypeptides represent another form of modified polypeptides that retain the important structural determinants for biological activity, yet eliminate the peptide bonds, thereby conferring resistance to proteolysis (Simon et al., Proc. Natl. Acad. Sci. USA 89:9367-9371, 1992, and incorporated herein by reference). Peptoids are oligomers of N-substituted glycines. A number of N-alkyl groups have been described, each corresponding to the side chain of a natural amino acid.

### Polynucleotides

The invention includes polynucleotides encoding peptides of the invention. Exemplary polynucleotides encode peptides including those described herein, and analogs, derivatives, amidated variations and conservative variations thereof, wherein the peptides have biological activity as described herein. The peptides of the invention include those described herein, as well as the broader groups of peptides having hydrophilic and hydrophobic substitutions, and conservative variations thereof.

“Isolated” when used in reference to a polynucleotide, refers to a polynucleotide substantially free of proteins, lipids, nucleic acids, for example, with which it is naturally associated. As used herein, “polynucleotide” refers to a polymer of deoxyribonucleotides or ribonucleotides, in the form of a separate fragment or as a component of a larger construct. DNA encoding a peptide of the invention can be assembled from cDNA fragments or from oligonucleotides which provide a synthetic gene which is capable of being expressed in a recombinant transcriptional unit. Polynucleotide sequences of the invention include DNA, RNA and cDNA sequences. A polynucleotide sequence can be deduced from the genetic code, however, the degeneracy of the code must be taken into account. Polynucleotides of the invention include sequences which are degenerate as a result of the genetic code. Such polynucleotides are useful for the recombinant production of large quantities of a peptide of interest, such as the peptides described herein.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

In the present invention, the polynucleotides encoding the peptides of the invention can be inserted into a recombinant “expression vector”. The term “expression vector” refers to a plasmid, virus or other vehicle known in the art that has been manipulated by insertion or incorporation of genetic sequences. Such expression vectors of the invention are preferably plasmids that contain a promoter sequence that facilitates the efficient transcription of the inserted genetic sequence in the host. The expression vector typically contains an origin of replication, a promoter, as well as specific genes that allow phenotypic selection of the transformed cells. For example, the expression of the peptides of the invention can be placed under control of E. coli chromosomal DNA comprising a lactose or lac operon which mediates lactose utilization by elaborating the enzyme beta-galactosidase. The lac control system can be induced by IPTG. A plasmid can be constructed to contain the lac Iq repressor gene, permitting repression of the lac promoter until IPTG is added. Other promoter systems known in the art include beta lactamase, lambda promoters, the protein A promoter, and the tryptophan promoter systems. While these are the most commonly used, other microbial promoters, both inducible and constitutive, can be utilized as well. The vector contains a replicon site and control sequences which are derived from species compatible with the host cell. In addition, the vector can carry specific gene(s) which are capable of providing phenotypic selection in transformed cells. For example, the beta-lactamase gene confers ampicillin resistance to those transformed cells containing the vector with the beta-lactamase gene. An exemplary expression system for production of the peptides of the invention is described in U.S. Pat. No. 5,707,855.

Transformation of a host cell with the polynucleotide can be carried out by conventional techniques known to those skilled in the art. For example, where the host is prokaryotic, such as E. coli, competent cells that are capable of DNA uptake can be prepared from cells harvested after exponential growth and subsequently treated by the CaCl2 method using procedures known in the art. Alternatively, MgCl2 or RbCl could be used.

In addition to conventional chemical methods of transformation, the plasmid vectors of the invention can be introduced into a host cell by physical means, such as by electroporation or microinjection. Electroporation allows transfer of the vector by high voltage electric impulse, which creates pores in the plasma membrane of the host and is performed according to methods known in the art. Additionally, cloned DNA can be introduced into host cells by protoplast fusion, using methods known in the art.

DNA sequences encoding the peptides can be expressed in vivo by DNA transfer into a suitable host cell. “Host cells” of the invention are those in which a vector can be propagated and its DNA expressed. The term also includes any progeny of the subject host cell. It is understood that not all progeny are identical to the parental cell, since there can be mutations that occur during replication. However, such progeny are included when the terms above are used. Preferred host cells of the invention include E. coli, S. aureus and P. aeruginosa, although other Gram-negative and Gram-positive organisms known in the art can be utilized as long as the expression vectors contain an origin of replication to permit expression in the host.

The polynucleotide sequence encoding the peptide used according to the method of the invention can be isolated from an organism or synthesized in the laboratory. Specific DNA sequences encoding the peptide of interest can be obtained by: 1) isolation of a double-stranded DNA sequence from the genomic DNA; 2) chemical manufacture of a DNA sequence to provide the necessary codons for the peptide of interest; and 3) in vitro synthesis of a double-stranded DNA sequence by reverse transcription of mRNA isolated from a donor cell. In the latter case, a double-stranded DNA complement of mRNA is eventually formed that is generally referred to as cDNA.

The synthesis of DNA sequences is frequently the method of choice when the entire sequence of amino acid residues of the desired peptide product is known. In the present invention, the synthesis of a DNA sequence has the advantage of allowing the incorporation of codons that are more likely to be recognized by a bacterial host, thereby permitting high level expression without difficulties in translation. In addition, virtually any peptide can be synthesized, including those encoding natural peptides, variants of the same, or synthetic peptides.

### Assays

The ability of a Bcl protein to inhibit cell death in a mammal, and/or to inhibit inflammation in a mammal, can be assessed, for example, in a mammal subjected to ischemia-reperfusion injury. For example, one or more of the following markers and/or assays can be used to assess the ability of a Bcl protein to inhibit cell death and/or inflammation in a mammal subjected to ischemia-reperfusion injury: 1 Inhibition of inflammation and/or cell death is shown by a reduction in creatine kinase concentration in the plasma or serum of a mammal after ischemia-reperfusion of skeletal muscle (see, e.g., Example 9 herein, and Iwata, A., et al., Blood 100:2077, 2002); 2. Inhibition of inflammation and/or cell death is shown by a reduction in infarct size following ischemia-reperfusion of mammalian heart or mammalian brain (see, e.g., Example 10 herein, and Palazzo, A. J., Am. J. Physiol. 275:H2300, 1998; Piot, C, Circulation 96:1598, 1997); 3 Inhibition of inflammation and/or cell death is shown by a reduction in blood urea nitrogen (BUN) and/or creatine following ischemia-reperfusion of mammalian kidney (see, e.g., Daemen, M., J. Clin. Invest. 104:541, 1999; Vukicevic, S., J. Clin. Invest. 102:202, 1998); 4. Inhibition of inflammation and/or cell death is shown by a reduction in aspartate aminotransferase (AST) and/or alanine aminotransferase (ALT) following ischemia-reperfusion of mammalian liver (see, e.g., Cursio, R., FASEB J. 13:253, 1999); 5. Inhibition of inflammation and/or cell death is shown by an improvement in arterial oxygen content following ischemia-reperfusion of mammalian lung; 6 Inhibition of inflammation and/or apoptosis is shown by a reduction in lung edema following ischemia-reperfusion of mammalian lung (see, e.g., Kowalski, T. F., J. Appl. Physiol. 68:125, 1990; 7 Inhibition of cell death is shown by a reduction in markers of cell death (e.g., by reduced DNA strand-breaks assessed by terminal deoxynucleotidyl transferase end labeling (TUNEL), by reduced caspase activation, by increased expression of phosphatidyl serine on the cell surface, by decreased DNA ladder of 180-200 base pair following electrophoresis) in tissue (e.g., skeletal muscle, heart, brain, lung, intestine, kidney, or liver) subjected to ischemia-reperfusion injury (see, e.g., Iwata, et al., Blood 100:2077, 2002; Piot, C., Circulation 96:1598, 1997; Namura, S., J. Neurosci. 18:3659, 1998; Noda, T., Am. J. Physiol. 274:G270, 1998; and Cursio, R., FASEB J. 13:253, 1999).

Inhibition of inflammation and/or cell death as a result of administration of a Bcl protein can also be assessed in a mammal subjected to sepsis (e.g., sepsis due to cecal ligation and puncture, sepsis due to bacterial pneumonia, sepsis due to bacterial peritonitis), or in a mammal receiving injections or infusions (e.g., injections or infusions into the peritoneum, injections or infusions into the lung, subcutaneous injections or infusions, intra-dermal injections or infusions) of substances that promote sepsis (e.g., lipopolysaccharide, bacterial lipoproteins, lipoteichoic acid). Inhibition of inflammation and/or cell death as a result of administration of a Bcl protein is indicated by increased survival in mammals following initiation of sepsis by the injection or infusion of substances that promote sepsis.

### Exemplary Treatment Regimes

In general, there are at least four goals in treating HF patients (1) treating the symptoms, (2) slowing the progression of cardiac dysfunction, (3) decreasing length of hospital stay, and (4) increasing the time between hospitalization, all while minimizing health care costs. It is believed that the methods of treating HF according to this invention will achieve one or more of these goals.

According to one aspect, this invention provides a method of treating HF in a patient comprising administering Bcl protein or a pharmaceutically acceptable composition thereof to the patient at a rate between about 50 and 500 ng/min for a time between 30 minutes and 8 hours per day for as many days as needed to provide symptomatic relief, prevent exacerbation of symptoms, and/or prevent and/or delay progression of the disease state of heart failure in said patient. For example, Bcl protein can be continuously or intermittently administered for a period of time between about 24 and 48 hours, or as a bolus dose. If Bcl protein is administered two or more times intermittently each day, lower doses, e.g., 0.8 to 10 ng/min can be administered.

Treatment is continued as needed to provide symptomatic relief, prevent exacerbation of symptoms, and/or prevent and/or delay progression of the disease state of heart failure in said patient, or until it is no longer well tolerated by the patient, or until a physician terminates treatment. For example, a physician can monitor one or more symptoms of HF, renal blood flow, glomerular filtration rates, and/or serum levels of urea and creatinine in a patient being treated with Bcl protein according to this invention and, upon observing attenuation of one or more symptoms of HF for a period of time, conclude that the patient can sustain the positive effects of the above-described treatment without further administration of Bcl protein for a period of time. If necessary, the patient can then return at a later point in time for additional treatment as needed.

According to another aspect, this invention provides a method of treating HF in a patient comprising administering Bcl protein to the patient at a rate between about 500 and 600 ng/min for period between about 8 hours per day for at least three consecutive days or several times per week as needed to provide symptomatic relief, prevent exacerbation of symptoms, and/or prevent and/or delay progression of the disease state of heart failure in the patient. This treatment can be provided as outpatient therapy to prevent exacerbation of the heart failure and to enhance the quality of life in the patient.

As used herein, “day” means a 24-hour period. Thus for example, “for at least three consecutive days” means for at least a 72-hour period. During or after the treatment, a physician can monitor one or more symptoms of HF, renal blood flow, glomerular filtration rates, and/or serum levels of urea or creatinine in the patient and, upon observing an improvement in one or more of the parameters for a period of time, conclude that the patient can sustain the positive effects of the treatment without further administration of Bcl protein for a period of time.

According to another aspect, this invention provides a method of treating HF in a patient comprising administering Bcl protein to the patient at a rate between about 50 and 400 ng/min over a period of up to 8 hours per day for each day of hospitalization of the patient or as needed. In certain cases the patient can require higher doses, for example up to 2 μg/min over the same time period.

Once treatment with Bcl protein according to any of the methods of this invention has achieved the desired results, e.g., symptomatic relief, prevent exacerbation of symptoms, and/or prevent and/or delay progression of the disease state of heart failure, the patient can then receive maintenance therapy if desired. For example, a lower dose of Bcl protein, e.g., less than 10 ng/min, can be administered to the patient for maintenance therapy by any suitable route including, but not limited to, injection, intravenous administration, and the like. In one aspect, the delivery regime can be designed to deliver between Bcl protein at a rate between about 0.8 to 10 ng/min for a desired period of time, such as over a period of 3, 6 or 9 months or more.

Because Bcl protein therapy according to any of the methods of this invention prevents further damage from ischemic injury and promotes the healing process, it can also be used to delay or preclude further exacerbation of a heart condition into a more serious and progressive diseases such as HF. Thus, each of the above-described methods can also be used as a prophylactic treatment to prevent or slow the progression of early stages of HF to more advanced stages. That is, once treatment with Bcl protein according to any of the methods of this invention has achieved the desired results, the patient can optionally receive maintenance therapy thereafter. For example, one aspect of this invention for providing maintenance therapy to a patient with a heart condition comprises providing a lower dose of Bcl protein, e.g., less than 10 ng/min, to the patient for maintenance therapy by any suitable route including, but not limited to, injection, intravenous administration, controlled release administration, and the like In one aspect, the delivery system can be designed to deliver Bcl protein at a rate between about 0.8 to 10 ng/min for a desired period of time, such as over a period of 3, 6 or 9 months or more. In an alternative aspect, the patient can receive long-term, low dose, maintenance administration of Bcl protein from a controlled release formulation.

In addition, it is known that a patient that has suffered a myocardial infarction (MI) will likely suffer another MI in the future. Thus, a patient having an MI can be treated with an initial dose of Bcl protein according to any of the methods of this invention until one or more symptoms of HF has diminished, and subsequently can be put on a Bcl protein maintenance dosing regime. The maintenance regime can also be given to a post-MI patient that was initially treated for MI by means other than Bcl protein, and can also be used for HF patients that have not yet suffered an MI as a means to slow the progression of HF into the more advanced stages or to prevent or reduce the risk of MI in patients with advanced HF.

The invention provides pharmaceutical compositions comprising one or more BCL2 peptides, for example, formulated together with a pharmaceutically acceptable carrier. Some compositions include a combination of multiple (e.g., two or more) peptides of the invention. Some compositions include a combination of a peptide and/or peptides of the invention together with other drugs or agents.

As used herein “pharmaceutically acceptable carrier” and “pharmaceutically acceptable excipient” include any and all solvents, dispersion media, coatings, antibacterial and antifungal agents, isotonic and absorption delaying agents, and the like that are physiologically compatible. In one aspect, the carrier is suitable for parenteral administration. Alternatively, the carrier can be suitable for intravenous, intraperitoneal or intramuscular administration. In another aspect, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is compatible with the active compound, use thereof in the pharmaceutical compositions is contemplated. Supplementary active compounds can also be incorporated into the compositions.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g. C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

“Pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

In prophylactic applications, pharmaceutical compositions or medicaments are administered to a patient susceptible to, or otherwise at risk of a disease or condition (i.e., by chronic heart failure, chronic heart failure at a phase of acute exacerbation, a phase of transition to chronic heart failure or the like) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease or condition in an amount sufficient to cure, or at least partially arrest, the symptoms of the disease or condition (e.g., biochemical and/or histologic), including its complications and intermediate pathological phenotypes in development of the disease or condition. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if the response starts to wane.

The pharmaceutical composition of the present invention should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

### Routes of Administration

A composition of the present invention can be administered by a variety of methods known in the art. The route and/or mode of administration vary depending upon the desired results. The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. The BCL2 peptide(s) of the invention can be administered parenterally by injection or by gradual infusion over time. The peptide can also be prepared with carriers that protect the compound against rapid release, such as a controlled release formulation, including implants, transdermal patches, and microencapsulated delivery systems. Further methods for delivery of the peptide include orally, by encapsulation in microspheres or proteinoids, by aerosol delivery to the lungs, or transdermally by iontophoresis or transdermal electroporation. To administer a peptide of the invention by certain routes of administration, it can be necessary to coat the compound with, or co-administer the compound with, a material to prevent its inactivation. The method of the invention also includes delivery systems such as microencapsulation of Bcl into liposomes or a diluent. Microencapsulation also allows co-entrapment of Bcl molecules along with the antigens, so that these molecules, such as antibiotics, can be delivered to a site in need of such treatment in conjunction with the Bcl of the invention. Liposomes in the blood stream are generally taken up by the liver and spleen. Pharmaceutically acceptable diluents include saline and aqueous buffer solutions. Liposomes include water-in-oil-in-water CGF emulsions as well as conventional liposomes. Strejan et al., J. Neuroimmunol. 7: 27, 1984. Thus, the method of the invention is particularly useful for delivering Bcl peptides to such organs. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Robinson, 1978, Sustained and Controlled Release Drug Delivery Systems. Other methods of administration will be known to those skilled in the art.

Preparations for parenteral administration of a peptide of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred aspect, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

When the Bcl protein sof the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

### Effective Dosages

“Therapeutically effective amount” as used herein for treatment of diseases and conditions refers to the amount of peptide used that is of sufficient quantity to decrease cell death and inflammation brought about by chronic heart failure, chronic heart failure at a phase of acute exacerbation, a phase of transition to chronic heart failure or the like. The dosage ranges for the administration of Bcl are those large enough to produce the desired effect. The amount of peptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's (Remington's Pharmaceutical Science, Mack Publishing Company, Easton, Pa.); Egleton, Peptides 18: 1431-1439, 1997; Langer, Science 249: 1527-1533, 1990. The dosage regimen can be adjusted by the individual physician in the event of any contraindications.

In some aspects, acute adminstration is contemplated to inhibit cell death and/or inflammation, an effective analgesic response eliciting dose, in most instances, will be about 5 mg to 500 mg as needed (e.g., every four to twenty-four hours). For chronic adminstration to alleviate (treat) (chronic) inflammation and pain, inhibit IL-1 biosyntheses and/or inhibit prostaglandin H.sub.2 synthase, in most instances, an effective dose will be from about 5 mg to 1.0 g per day, and preferably 50 mg to 500 mg per day, in single or divided doses. On the other hand, it may be necessary to use dosages outside these limits in some cases.

Dosage regimens of the pharmaceutical compositions of the present invention are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compound of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic composition can be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

An effective dose of each of the Bcl proteins disclosed herein as potential therapeutics for use in treating microbial diseases and conditions is from about 1 μg to 500 mg/kg body weight, per single administration, which can readily be determined by one skilled in the art. As discussed above, the dosage depends upon the age, sex, health, and weight of the recipient, kind of concurrent therapy, if any, and frequency of treatment. Other effective dosage range upper limits are 100 mg/kg body weight, 50 mg/kg body weight, 25 mg/kg body weight, and 10 mg/kg body weight.

The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Exemplary Bcl dosages include administration of at least 50 ng/kg/day, such as from 50 ng/kg/day to 50 mg/kg/day, or such as from 0.5 mg/kg/day to 50 mg/kg/day, for a period of time sufficient to inhibit cell death and/or inflammation in the mammal brought about by heart failure. Typically, the Bcl protein is administered to the mammal on multiple occasions (e.g., daily). For example, a Bcl protein can be administered to a mammal at least once per day for a period of from 1 day to 20 days, or from 1 day to 40 days, or from 1 day to 60 days. Bcl protein can be administered indefinitely to a mammalian subject to treat a chronic medical condition such as chronic heart failure (e.g., at least once per day each day during the remaining lifetime of the recipient). The abbreviation “ng” is an abbreviation for nanogram, or nanograms, as appropriate. The abbreviation “mg” is an abbreviation for milligram, or milligrams, as appropriate. The abbreviation “kg” is an abbreviation for kilogram, or kilograms, as appropriate.

On the other hand, it may be necessary to use dosages outside these limits. For example, for acute administration to alleviate (treat) cell death and inflammation brought about by a variety of diseases and conditions including heart failure (i.e., acute heart failure, chronic heart failure, chronic heart failure at a phase of acute exacerbation or heart failure at a phase of transition to chronic heart failure), an effective response eliciting dose, in most instances, is about 5 mg to 500 mg as needed (e.g., every four to twenty-four hours).

For chronic administration to alleviate (treat) cell death and inflammation brought about by a variety of diseases and conditions including heart failure (i.e., acute heart failure, chronic heart failure, chronic heart failure at a phase of acute exacerbation or heart failure at a phase of transition to chronic heart failure), an effective dose will be from about 5 mg to 1.0 g per day, and preferably 50 mg to 500 mg per day, in single or divided doses.

Some compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, See, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., Ranade, J. Clin. Pharmacol. 29: 685, 1989). Exemplary targeting moieties include folate or biotin (See, e.g., U.S. Pat. No. 5,416,016 to Low et al.,); mannosides (Umezawa et al., Biochem. Biophys. Res. Commun. 153: 1038, 1988); antibodies (Bloeman et al., FEBS Lett. 357: 140, 1995; Owais et al., Antimicrob. Agents Chemother. 39: 180, 1995); surfactant protein A receptor (Briscoe et al., Am. J. Physiol. 1233: 134, 1995), different species of which can comprise the formulations of the inventions, as well as components of the invented molecules; p120 (Schreier et al., J. Biol. Chem. 269: 9090, 1994); See also Keinanen et al., FEBS Lett. 346: 123, 1994; Killion et al., Immunomethods 4: 273, 1994. In some methods, the therapeutic compounds of the invention are formulated in liposomes; in a more preferred aspect, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

If necessary, Bcl protein can be administered according to the methods of this invention either alone or in combination with at least one other agent including, but not limited to, anti-proliferative agents, anti-clotting agents, vasodilators, diuretics, beta-blockers, calcium ion channel blockers, blood thinners, cardiotonics, ACE inhibitors, anti-inflammatories, antioxidants, and/or gene therapeutics. When used in combination with other agents, Bcl protein and the agent can be administered separately (either simultaneously or separately in any order) or in admixture. In one aspect, when Bcl protein and at least one other agent are administered as separate components, they are administered to the patient at about the same time. “About the same time” means that within about thirty minutes of administering one compound (e.g., Bcl protein) to the patient, the other compound (e.g., an anti-proliferative or anti-clotting agent) is administered to the patient. “About the same time” also includes concomitant or simultaneous administration of the compounds.

### Formulation

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### Kits

For use in research and therapeutic applications suggested above, kits are also provided by the invention. In the diagnostic and research applications such kits can include any or all of the following: assay reagents, buffers BCL proteins, hybridization probes and/or primers, BCL variant polypeptides or polynucleotides, and the like. A therapeutic product can include sterile saline or another pharmaceutically acceptable emulsion and suspension base as described above.

Accordingly, kits of the present invention can contain any reagent used to treat diseases and disorders as described herein. Kits of the present invention can also contain additional agents that can be administered concomitantly with the compositions of the present invention.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

From the foregoing description, various modifications and changes in the compositions and methods will occur to those skilled in the art. All such modifications coming within the scope of the appended claims are intended to be included therein. Each recited range includes all combinations and sub-combinations of ranges, as well as specific numerals contained therein.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

### Exemplary Aspects

### Example 1

This Example describes expression of a cDNA (SEQ ID NO:13) that encodes the anti-apoptotic protein, human Bcl-2 (SEQ ID NO:14) in myeloid cells. Expression of the Bcl-2 protein (SEQ ID NO:14) in myeloid cells reduced injury following extended ischemia in the mouse hind limb.

Mice expressing a recombinant human Bcl-2 (SEQ ID NO:14) in their myeloid cells (hMRP8-myeloid-Bcl-2 mice), and control mice that did not express the recombinant human Bcl-2 (SEQ ID NO:14) in their myeloid cells were used in this experiment. The hMRP-myeloid-Bcl-2 mice were previously described by Lagasse, E., and I. L. Weissman, J. Exp. Med. 179:1047, 1994, which publication is incorporated herein by reference. These mice were on a C57BL/6 background, and the control mice were C57BL/6 mice.

Mouse skeletal muscle was made ischemic by cross-clamping the aorta distal to the renal artery for 90 minutes, then the clamp was removed and hind limb reperfusion continued for 3 hours. At the end of reperfusion (3 hours after clamp removal) the mice were killed, and the concentration of plasma creatine kinase (CK) was measured and used as an indicator of injury (creatine kinase concentration increases as a result of skeletal muscle injury).

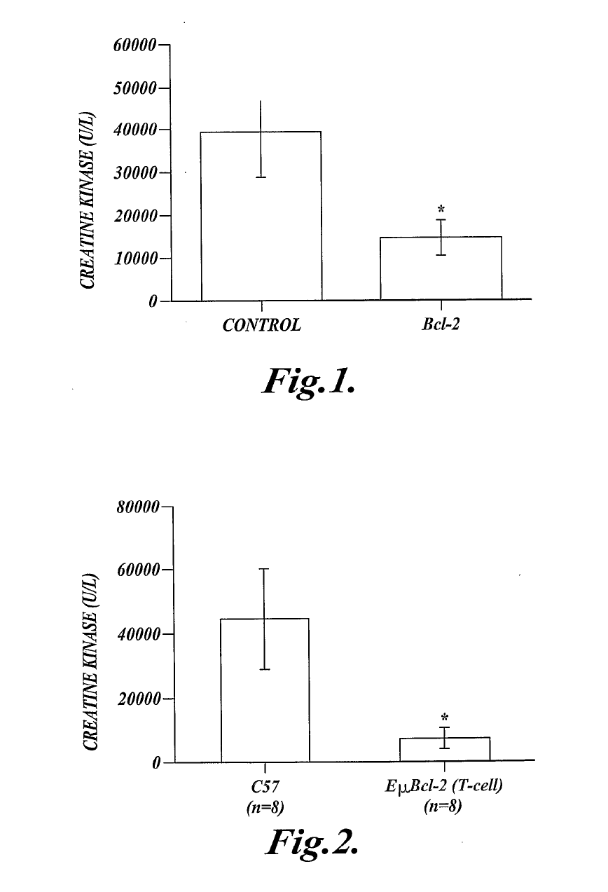

The results of these experiments are shown in FIG. 1. The plasma creatine kinase levels were significantly less in the eleven hMRP8-myeloid-Bcl-2 mice (designated Bcl-2) compared to the nine control C57BL/6 mice (*p<0.05), suggesting that human Bcl-2 (SEQ ID NO:14) protects the mice from ischemia-reperfusion injury. It has been reported in the literature, however, that neutrophils from the hMRP8-myeloid-Bcl-2 mice exhibit reduced apoptosis (Lagasse, E., and Weissman, I. L., J. Exp. Med. 179:1047, 1994), and it is possible that neutrophil apoptosis contributes to ischemia-reperfusion injury by release of toxic products at the site of injury. Thus, it is possible that Bcl-2 is preventing apoptosis of neutrophils, thereby reducing the amount of ischemia-reperfusion injury in the mice that express Bcl-2 in their myeloid cells.

It is unlikely, however, that neutrophils are involved in the ischemia-reperfusion injury for the extended ischemia time of 90 minutes since blocking their emigration into tissue with anti-CD18 mAb has no effect on ischemia-reperfusion injury (Iwata, A. et al., Blood, 100:2077 (2002)). Moreover, as shown in Example 2, over-expression of human Bcl-2 (SEQ ID NO:13) in T-lymphocytes also protects against ischemia-reperfusion injury. Thus, it appears to be unlikely that over-expression of human Bcl-2 in myeloid cells is protecting muscle by preventing apoptosis of leukocytes.

### Example 2

This example shows that over-expression of human Bcl-2 (SEQ ID NO:14) in T-lymphocytes reduces skeletal muscle injury following extended ischemia in the mouse hind limb.

Transgenic mice, on a C57BL/6 genetic background, expressing exogenous human Bcl-2 (SEQ ID NO:14) in their T-cells under the control of the Eμ-promoter (EμT-Bcl-2 mice), and eight C57BL/6 control mice that did not express exogenous Bcl-2 (SEQ ID NO:14) in their T-cells (C57BL/6 mice) were used in this experiment. The EμT-Bcl-2 mice have been previously described and shown to express Bcl-2 (SEQ ID NO:14) only in T-lymphocytes (Strasser, A., et al., Cell 67:889, 1991, which publication is incorporated herein by reference).

Hind limb ischemia was induced by cross clamping the aorta as described in Example 1, and ischemia was maintained for 90 minutes followed by 3 hours of reperfusion. Blood samples were taken at the end of the experiment for determination of creatine kinase (CK) concentration in EμT-Bcl-2 mice and C57B1/6 mice. As shown in FIG. 2, serum CK in the eight EμT-Bcl-2 mice was significantly less than in the eight C57BL/6 control mice (designated C57) (*p<0.05).

### Example 3

This example shows that over-expression of Bcl-2 (SEQ ID NO:14) in leukocytes reduces DNA strand-breaks in skeletal muscle following extended ischemia and reperfusion of the hind limb.

Transgenic mice (described in Example 2) expressing exogenous human Bcl-2 (SEQ ID NO:14) in their T-cells under the control of the Eμ-promoter (EμT-Bcl-2 mice); transgenic mice expressing exogenous human Bcl-2 (SEQ ID NO:14) in their B-cells under the control of the Eμ-promoter (EμB-Bcl-2 mice) (reported in Strasser, A., Proc. Natl. Acad. Sci. 88:8661, 1991); transgenic mice (described in Example 1) expressing exogenous human Bcl-2 (SEQ ID NO:14) in their myeloid cells (hMRP8-myeloid-Bcl-2 mice); and control mice (C57BL/6 mice) that did not express exogenous Bcl-2 (SEQ ID NO:14), were used in this experiment.

It is known that extended ischemia followed by reperfusion of skeletal muscle results in DNA strand-breaks, and that treatment with the caspase inhibitor z-VAD prevents the strand-breaks and reduces the plasma CK concentration (Iwata, A., et al., Blood 100:2077, 2002). Tissue from the legs of control mice, EμT-Bcl-2 mice, EμB-Bcl-2 mice and hMRP8-myeloid-Bcl-2 transgenic mice was fixed in formalin and stained to identify DNA strand breaks using the TUNEL technique as described by the manufacturer (In Situ Cell Death Detection Kit, Roche Applied Science, PO Box 50414, 9115 Hague Road, Indianapolis, Ind. 46250-0414).

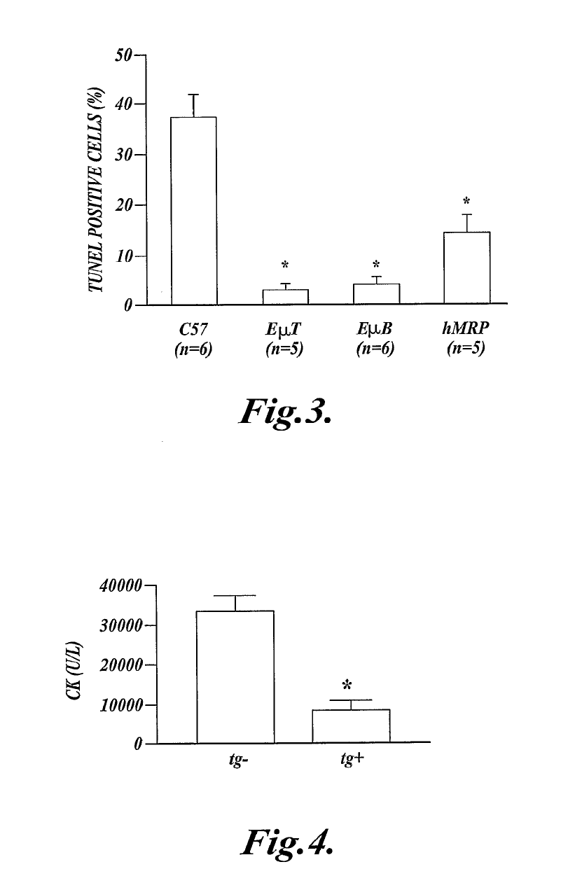

The number of nuclei that stained positive (indicating the presence of DNA strand breaks) as a percent of the total number of nuclei is shown in FIG. 3 for all four types of mice. The control C57BL/6 mice (designated C57) had significantly more positive nuclei compared with each of the transgenic strains (*p<0.05). In the mice that expressed Bcl-2 (SEQ ID NO:14) in one cell type (EμB in B cells, EμT inT cells, or hMRP8 in myeloid cells) DNA strand breaks were prevented in skeletal muscle and endothelial cells, other than the cells that expressed the Bcl-2 (SEQ ID NO:14), suggesting that the cells that expressed Bcl-2 released a molecule that protected cells from DNA strand breaks. That is, protection occurs as a “trans” effect.

### Example 4

This example shows that blood plasma from mice over-expressing hBcl-2 (SEQ ID NO:14) in T-lymphocytes reduces injury following extended ischemia followed by reperfusion.

Transgenic mice (described in Example 2) expressing exogenous human Bcl-2 (SEQ ID NO:14) in their T-lymphocytes (EμT-Bcl-2 mice), and littermate control mice that did not express exogenous human Bcl-2 (SEQ ID NO:14) (C57B1/6 mice) were used in this experiment. Blood from EμT-Bcl-2 mice and their littermate control mice was drawn into heparin and plasma was extracted by centrifugation. One ml of plasma from EμT-Bcl-2 mice, or one ml of plasma from littermate control mice, was injected into the peritoneum of C57BL/6 mice the day before the mice were subjected to 90 minutes of ischemia and 3 hours of reperfusion as described in Example 1 herein. Blood was drawn from the mice and plasma CK concentration was determined at the end of reperfusion. The results of these experiments are shown in FIG. 4. The six mice that received an injection of plasma from the EμT-Bcl-2 mice (designated tg+) had significantly lower concentrations of CK compared with the six mice that received an injection of plasma from littermate control mice (designated tg−) that did not express exogenous Bcl-2 (SEQ ID NO:14) (*p<0.05).

These results show that over-expression of Bcl-2 (SEQ ID NO:14) in the hMRP8-Bcl-2 mice results in the release of a molecule, that acts in “trans”, that can be transferred to naive, control, recipient mice and that protects the recipient mice from ischemia-reperfusion injury.

### Example 5

This example shows that injection of transgenic Jaws II leukocyte cells, that express a cDNA (SEQ ID NO:13) encoding a Bcl-2 protein (SEQ ID NO:14), into mice reduced the amount of ischemia-reperfusion injury compared to control mice that were not injected with Jaws II leukocyte cells.

The recent generation of high efficiency retroviral packaging cell lines, coupled with the development of retroviral expression vectors containing internal ribosome entry site (IRES) elements that allow the expression of two genes from a single mRNA transcript, has provided a new tool for gene transfer into mammalian cells (see, e.g., Hitoshi, Y., et al., Immunity 8:461, 1998; Onishi, M., et al., Exp. Hematol. 24:324 1996).

A cDNA (SEQ ID NO:13) encoding a hBcl-2 (SEQ ID NO:14) was inserted upstream of the IRES site into the pBM-IRES-EGFP retroviral vector, in which the cDNA for the enhanced green fluorescent protein (EGFP) is cloned downstream of the IRES sequence to produce the pBM-hBcl-2-IRES-EGFP vector. The ecotropic packaging cell line Phoenix was used to produce viral particles to transfect Jaws II cells that were obtained from ATCC. The Phoenix cell line and the retroviral vector are described by Hitoshi, Y., et al., Immunity 8:461, 1998, and by Onishi, M., et al., Exp. Hematol. 24:324, 1996.

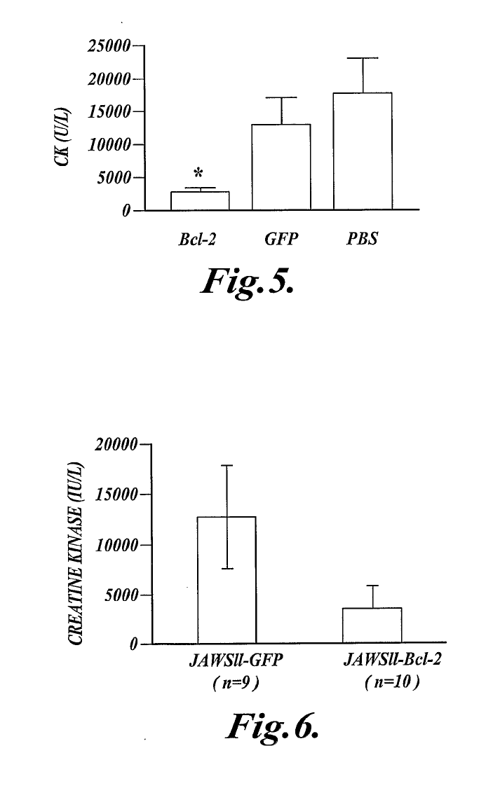

Control Jaws II cells expressed only EGFP. The hBcl-2-JawsII cells or EGFP-JawsII cells were sorted for cells with high expression of EGFP, then injected into C57BL/6 mice the day before subjecting the mice to extended ischemia-reperfusion. As shown in FIG. 5, the 18 mice injected with the hBcl-2-JawsII cells (designated Bcl-2) had significantly lower plasma CK concentration compared with either the 12 mice injected with phosphate buffered saline (designated PBS), or the 16 mice injected with EGFP-JawsII cells (designated GFP). (*p<0.05)

### Example 6

This example shows that the blood plasma CK concentration in mice subjected to ischemic reperfusion injury was lower in mice that had been injected with supernatant from an in vitro culture of hBcl-2-Jaws II cells (that express Bcl-2 (SEQ ID NO:14)), compared to the blood plasma CK concentration in mice that had been injected with supernatant from an in vitro culture of Jaws II cells that expressed enhanced green fluorescent protein (EGFP, EGFP-JawsII cells). The hBcl-2-Jaws II cells and the EGFP-Jaws II cells are described in Example 5.

Medium from cell cultures of hBcl-2-Jaws II cells and EGFP-Jaws II cells was harvested 24 hours after the start of incubation and concentrated approximately 10-fold using centrifugal filters with a molecular size cut-off of approximately 3 kD. One ml of concentrated medium from either hBcl-2-Jaws II cells or EGFP-Jaws II cells was injected into the peritoneum of C57BL/6 mice 24 hours prior to extended ischemia-reperfusion. Plasma was extracted from the mice after ischemia-reperfusion, and plasma CK concentration was determined As shown in FIG. 6, the plasma creatine kinase concentration in ten mice injected with hBcl-2-Jaws II cell supernatant (designated JAWSII-Bcl-2) was significantly less (p<0.05) than the plasma creatine kinase concentration in nine mice injected with EGFP-Jaws II cell supernatant (designated JAWSII-GFP).

### Example 7

This example shows that human Bcl-2 (SEQ ID NO:14) is secreted from cultured

JawsII-Bcl-2 cells into the culture supernatant.

Jaws II cells (described in Example 5) expressing either human Bcl-2 (SEQ ID NO:14) or enhanced green fluorescent protein (EGFP) were incubated for 24 hours in serum free medium, and the supernatants were separately collected and concentrated approximately 10 fold. Cells were disrupted in a protease inhibitor cocktail then the lysate and cell supernatant were subjected to immunoblot analysis for Bcl-2. Both the concentrated culture supernatant and the lysate from JawsII-Bcl-2 contained Bcl-2 protein, whereas no Bcl-2 protein was detected in either the concentrated culture supernatant or lysate of JawsII-GFP cells.

### Example 8

This Example shows that plasma creatine kinase levels in mice that had been subjected to hind leg ischemia and reperfusion was significantly lower in mice that were injected with modified, recombinant, Bcl-2 (SEQ ID NO:15) before hind leg ischemia and reperfusion, compared to the plasma creatine kinase levels in mice that were not injected with recombinant Bcl-2 before hind leg ischemia and reperfusion. SEQ ID NO:15 is a human Bcl-2 that lacks 17 amino acids at the carboxy terminal, and includes a series of 10 histidine residues on the carboxy terminal

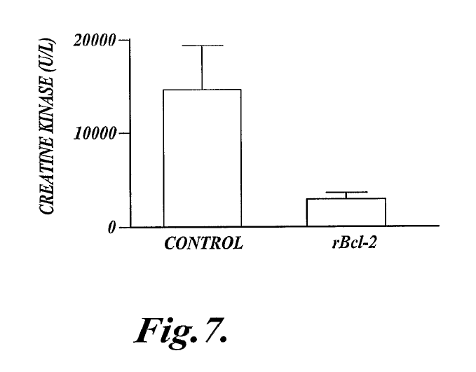

C57BL/6 mice were injected intraperitoneally with 1 μg per mouse of recombinant human Bcl-2 (rBcl-2) (SEQ ID NO:15) or 1 μg per mouse of recombinant human ubiquitin (rUbiquitin), or the vehicle solution for rBcl-2 the day before the mice were subjected to hind limb ischemia (90 minutes) and reperfusion (180 minutes) as described in Example 1. Blood samples were taken after the 90 minutes of hind limb ischemia and 180 minutes of reperfusion for determination of plasma creatine kinase concentration. There was no difference in the creatine kinase concentration between the two controls (rUbiquitin and vehicle solution) and these data were combined. As shown in FIG. 7, the plasma creatine kinase levels in mice that had been subjected to hind leg ischemia and reperfusion were significantly (p<0.05) lower in the 12 mice that were injected with recombinant human Bcl-2 (SEQ ID NO:15) (designated rBcl-2) before hind leg ischemia and reperfusion, compared to the plasma creatine kinase levels in the12 mice that received rUbiquitin or vehicle solution (designated CONTROL).

### Example 9

This example shows that over-expression of human Bcl-2 (SEQ ID NO:14) under a myeloid-restricted promoter reduces cardiomyocyte injury following extended ischemia in the mouse heart.

In order to determine whether over-expression of a Bcl-2 protein in leukocytes was protective in tissue other than skeletal muscle, myocardial ischemia-reperfusion injury was examined using ischemia times that were known to be CD18-independent (Palazzo, A. J., et al., Am. J. Physiol. 275:H2300, 1998). Control C57BL/6 and hMRP8-Bcl-2 mice were anesthetized, their trachea intubated, and they were placed on mechanical ventilation. A left thoracotomy was performed then an 8-0 suture was passed under the left anterior descending coronary artery (LAD) 2-3 mm from the tip of the left auricle, and the vessel was occluded. Care was taken not to damage the vessel. Occlusion was confirmed visually by change in color. The ligature was carefully removed after 1 hour of occlusion and reperfusion verified by direct visualization as color was re-established. The chest was closed taking care to remove air from the chest, the animal was extubated, given 0.5 ml of warmed saline, and placed in a heated incubator. Two hours later the mice were re-anesthetized, their trachea intubated, and they were placed on mechanical ventilation. The heart was exposed through the original incision and the original 8-0 suture re-tied. The mice were killed by exsanguination and a clamp was placed across the aorta, then 1 ml of 1.5% Evans Blue dye was injected by inserting a 30 gauge needle into the aorta so that the coronary circulation was perfused with dye.

The heart was removed, cut perpendicular to the long axis resulting in 4 sections that were incubated in 5 ml of 1% triphenyltetrazolium chloride (TTC) for 30 minutes. The left ventricle was placed in 10% buffered formaldehyde solution overnight following the removal of both the atrium and the right ventricle. Each heart slice was weighed then visualized under a microscope equipped with a CCD camera. The infarct area (uncolored), area at risk (AAR) (uncolored region plus brick red region) and total left ventricular region (AAR plus Evans Blue stained region) were measured by planimetry. The volume of infarction was estimated by the following equation:

V_{infarct}=A₁W₁+A₂W₂+A₃W₃+A₄W₄

Where A1, A2, A3, and A4 are the percent area of infarction in section 1, 2, 3, and 4, respectively and W1, W2, W3, and W4 are the corresponding weight in section 1, 2, 3, and 4, respectively. The volume at risk was calculated in a similar manner using appropriate areas.

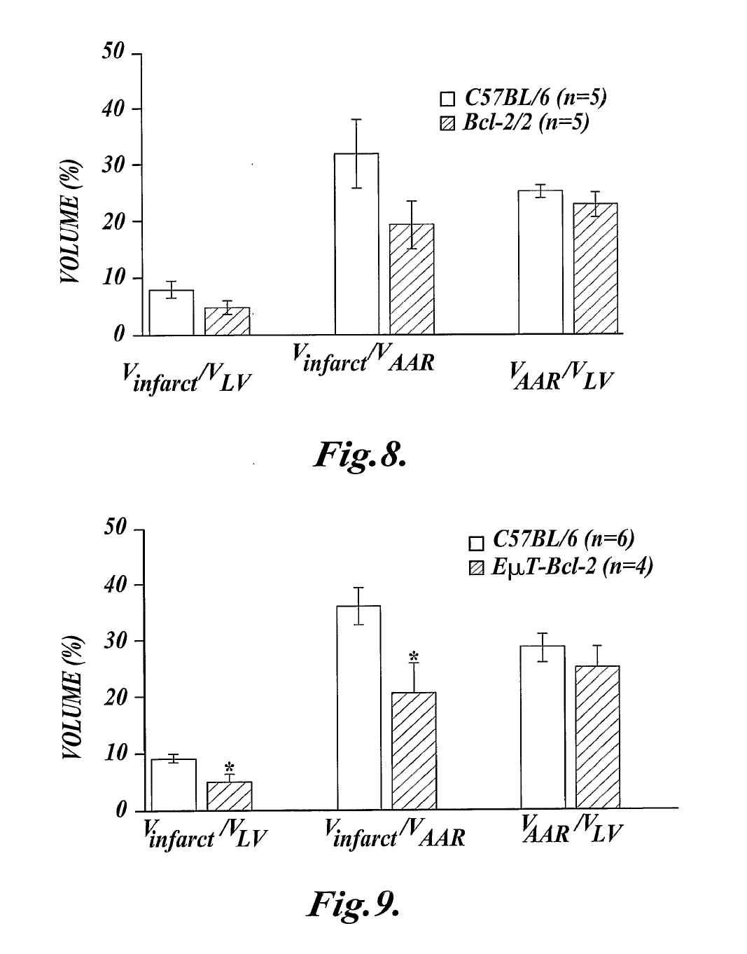

FIG. 8 shows the infarct volume as a percentage of left ventricular volume and as a percentage of the area at risk volume. The five hMRP8-Bcl-2 mice (designated Bcl-2/2) had reduced infarct volume by both measures compared with the five C57BL/6 control mice, and there were no differences in volume at risk to left ventricular volume between these two groups. V_{infarct}/V_{LV} and V_{infarct}/V_{AAR} of hMRP8-Bcl-2 mice were significantly reduced compared to C57BL/6 (p<0.05). There was no difference in volume at risk to left ventricular volume (V_{AAR}/V_{IN}) between the two groups.

### Example 10

This example shows that transgenic mice that express exogenous human Bcl-2 (SEQ ID NO:14) in their T-lymphocytes suffer less cardiomyocyte damage, caused by ischemia followed by reperfusion, than control mice that do not express exogenous Bcl-2 protein (SEQ ID NO:14) in their T-lymphocytes.

Additional myocardial ischemia-reperfusion experiments were performed using EμT-Bcl-2 mice that over-expressed Bcl-2 (SEQ ID NO:14) in their T-lymphocytes under the control of the Eμ promoter, and C57BL/6 control mice. The experiments were performed as described in Example 9, with coronary artery occlusion for 1 hour followed by 2 hours of reperfusion. Infarct volume (V_{infarct}) was calculated as a percent of left ventricle volume (V_{LV}), or as a percent of the volume of the area at risk (V_{AAR}). As shown in FIG. 9, V_{infarct}/V_{LV} and V_{infarct}/V_{AAR} were significantly reduced in the Eμ-T-lymphocyte-Bcl-2 mice versus C57BL/6 mice. (*p<0.05). There was no difference in volume at risk to left ventricular volume (V_{AAR}/V_{IN}) between the two groups.

### Example 11

This example shows that adoptive transfer of myeloid cells that express exogenous Bcl-2 protein (SEQ ID NO:14) reduces cardiomyocyte injury following extended ischemia in the mouse heart.

hMRP8-myeloid-Bcl-2 mice and littermate control mice were anesthetized, killed and bone marrow was extracted from their long bones. CD11b+ cells in the extracted bone marrow were isolated using magnetic beads (Miltenyi Biotec, 12740 Earhart Avenue, Auburn, Calif. 95602, USA) as described by the manufacturer. Approximately 10⁷ of these cells were administered to C57BL/6 control mice by intra-peritoneal injection 18 to 24 hours prior to hind limb ischemia and reperfusion. The ischemic period was 1 hour followed by 2 hours of reperfusion. Determination of infarct size was completed using the same technique as described in Example 9.

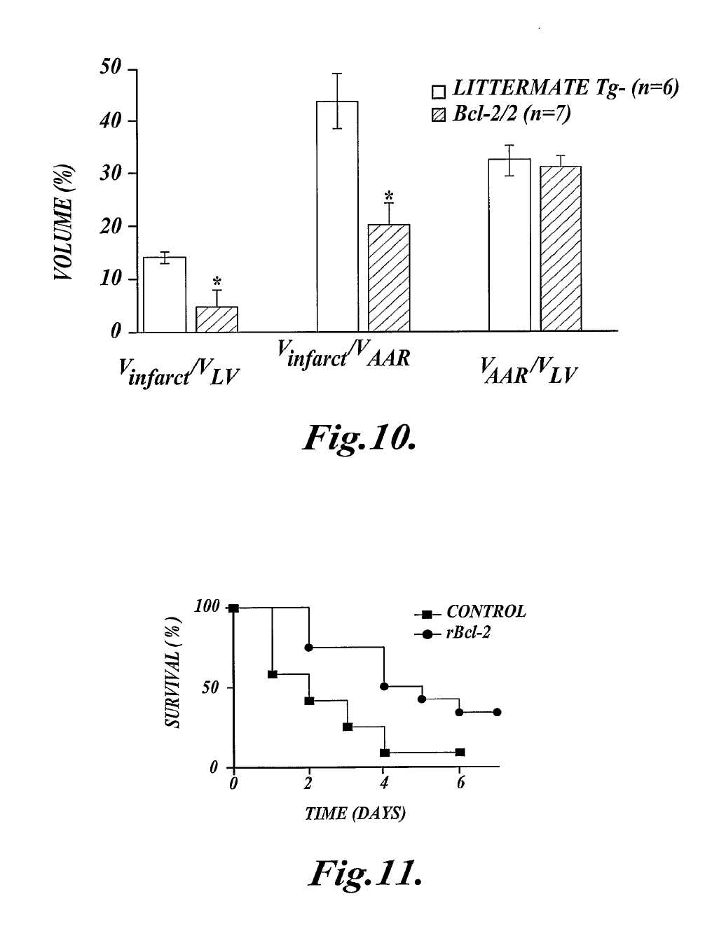

FIG. 10 shows the infarct volume as a percentage of left ventricular volume and as a percentage of the area at risk volume. The seven mice receiving bone marrow cells from the hMRP8-myeloid-Bcl-2 mice (designated Bcl-2/2) had significantly (*p<0.05) reduced infarct volume by both measures (V_{infarct/V}_{LV} and V_{infarct}V_{AAR}) compared with the six mice that received bone marrow cells from littermates (designated littermate Tg−). There was no difference in volume at risk to left ventricular volume (V_{AAR}/V_{LV}) between the two groups.

### Example 12

This example shows that over-expression of Bcl-2 provides protection in septic mice by a “trans” effect.

The survival of transgenic mice that expressed exogenous Bcl-2 (SEQ ID NO:14) in myeloid cells, under control of the human MRP8 promoter (hMRP8-Bcl-2 mice), or in T lymphocytes, under control of the Eμ promoter (EμT-Bcl-2 mice), was compared with the survival of C57BL/6 control mice following cecal ligation and puncture (CLP). 100% of hMRP8-Bcl-2 mice survived CLP, whereas only 25% of control mice survived CLP (p<0.05). In a separate experiment, 87.5% of EμT-Bcl-2 mice survived CLP, whereas only 22.2% of control mice survived CLP (p<0.05).

CD11b-positive bone marrow cells from hMRP8-Bcl-2 mice, or from C57BL/6 mice, were introduced into C57BL/6 mice, which were then subjected to CLP. 100% of the mice that received CD11b-positive bone marrow cells from hMRP8-Bcl-2 mice survived CLP, while none of the mice that received CD11b-positive bone marrow cells from C57BL/6 mice survived CLP.

CD11b-positive bone marrow cells from hMRP8-Bcl-2 mice, or from C57BL/6 mice, were introduced into Rag-1−/− mice (that did not have any mature T or B cells), which were then subjected to CLP. 87.5% of the mice that received CD11b-positive bone marrow cells from hMRP8-Bcl-2 mice survived CLP, while 12.5% of the mice that received CD11b-positive bone marrow cells from C57BL/6 mice survived CLP (p<0.05).

These experiments show that expression of hBcl-2 (SEQ ID NO:14) is protective in CLP and that protection is independent of lymphocytes.

### Example 13

This example shows that intraperitoneal injection of recombinant human Bcl-2 (SEQ ID NO:15) improves survival in mice subjected to severe sepsis as a result of cecal ligation and puncture.

Eight C57BL/6 mice were given an intraperitoneal injection of 1 μg per mouse recombinant human Bcl-2 and eight C57BL/6 mice were given and intraperitoneal injection of 1 μg per mouse recombinant human ubiquitin 12-24 hours prior to being subjected to cecal ligation and puncture as described in Example 12. An additional four C57BL/6 mice were given a subcutaneous injection of 10 μg of a maltose binding protein-hBcl-2 fusion protein and four C57BL/6 mice were given saline treatment 12-24 hours prior to being subjected to cecal ligation and puncture as described in Example 12. The mice were treated with antibiotics twice daily and with an additional treatment of recombinant human Bcl-2 or recombinant human ubiquitin or saline given daily for 3 days. Examination was conducted for signs of irreversible sepsis twice daily for 10 days through the use of a quantitative assessment form, and the mice were killed if they were deemed to be suffering from irreversible sepsis. FIG. 11 is a survival curve based on the results of these experiments and clearly shows that the 12 mice treated with recombinant human Bcl-2 (designated rBcl-2) had significantly (p<0.05) improved survival compared to the 12 mice treated with either recombinant human ubiquitin or saline (designated CONTROL).

### Example 14

This example shows that plasma creatine kinase levels in mice that had been subjected to hind leg ischemia and reperfusion was significantly lower in mice that were injected with modified recombinant human A1 (human A1 minus 25 amino acids at the carboxy terminal and addition of 6 histidine on the remaining protein) (SEQ ID NO:16) before hind leg ischemia and reperfusion, compared to the plasma creatine kinase levels in mice that were injected with recombinant ubiquitin before ischemia and reperfusion. Recombinant human A1 (SEQ ID NO:16) is a Bcl-2 protein.

C57BL/6 mice were injected intraperitoneally with recombinant human A1 (rA1) (SEQ ID NO:16) or recombinant human ubiquitin (rUbiquitin) the day before the mice were subjected to hind limb ischemia (90 minutes) and reperfusion (180 minutes) as described in Example 1. Blood samples were taken after the 180 minutes of reperfusion for determination of plasma creatine kinase concentration. The plasma creatine kinase levels in mice that had been subjected to hind leg ischemia and reperfusion were significantly (p<0.05) lower in the 12 mice that were injected with rA1 (SEQ ID NO:16) before hind leg ischemia and reperfusion, compared to the plasma creatine kinase levels in 12 control mice that were injected with rUbiquitin.

### Example 15

This example shows that fragments of a BH4 domain protected the hind-limbs of C57BL/6 mice from ischemia-reperfusion injury.

Skeletal muscle was made ischemic by applying a tourniquet to the hind-limbs of C57BL/6 mice for 90 minutes, then removing the tourniquet and allowing reperfusion for an additional 3 hours. Mice were treated with active peptide 1 (SEQ ID NO:17) or peptide 2 (SEQ ID NO:18) or peptide 3 (SEQ ID NO:19), or with the scrambled (control) peptide (SEQ ID NO:20). Peptide-1 (SEQ ID NO:17) is from the BH4 region of Bcl-2. Peptide-2 (SEQ ID NO:18) is from the first alpha helix of A1. Peptide-3 (SEQ ID NO:19) is from the BH4 region of Bcl-XL. The amino acid sequences of peptide 1 (SEQ ID NO:17), peptide 2 (SEQ ID NO:18), peptide 3 (SEQ ID NO:19), and the scrambled (control) peptide (SEQ ID NO:20) are set forth in Table 1.

TABLE 1

| | | |
|---|---|---|
| TGYDNREIVMKYIHYKLSQRGYEWD | peptide-1 | (SEQ ID NO: 17) |
| FGYIYRLAQDYLQCVLQIPZPGSGP | peptide-2 | (SEQ ID NO: 18) |
| MSQSNRELVVDFLSYKLSQKGYSWS | peptide-3 | (SEQ ID NO: 19) |
| QF | | |
| TWHMYGNQRDYIGDRSKIVYKLEYE | scrambled | (SEQ ID NO: 20) |
| | peptide | |

At the end of the 3 hours of reperfusion the mice were killed, and blood was taken for determination of plasma creatine kinase (CK) concentration. The CK concentration was used as an indicator of muscle injury. Elevated levels of CK indicate higher levels of muscle injury. Results from 3 separate experiments showed significant protection compared with the control peptide (p<0.05). The CK concentration for peptide 1 (SEQ ID NO:17) treated mice was 19,020+/−5481 IU/L (n=12) compared with control peptide (SEQ ID NO:20) where CK concentration was 60,530+/−5759 IU/L (n=12). The CK concentration for peptide 2 (SEQ ID NO:18) treated mice was 33,860+/−5997 IU/L (n=11) compared with control peptide (SEQ ID NO:20) where CK concentration was 69,430+/−11,170 IU/L (n=12). The CK concentration for peptide 3 (SEQ ID NO:19) treated mice was 49,500+/−3,901 IU/L (n=5) compared with control peptide (SEQ ID NO:20) where CK concentration was 80,880+/−11,430 IU/L (n=6).

### Example 16

Mice Heart Failure—Aorta Banding

Mice will be pretreated with buprenorphine, anesthetized with isoflurane, their trachea intubated and attached to a ventilator. An incision will be made in the chest wall approximately at the third intercostal space. A murine rib spreader will be inserted and the ribs gently spread to allow access to the thoracic cavity. The ascending aorta will be then isolated from the pulmonary artery and a sterile 7.0 silk ligature will be passed around it approximately 3 mm from the base of the heart. A blunted 27 gauge needle will be placed on top of the aorta and a ligature will be tied around the needle. The needle is then carefully removed from under the tie. The rib spreader removed, the chest closed and the lung re-inflated. The ribs and chest musculature are sutured using sterile 5.0 vicryl suture, while the skin is closed with 5-0 monofilament and the mice allowed to recover from anesthesia. Analgesics will be administered following the operation. Mice will be treated at various times. Specifically, mice will be divided into treatment groups. Group 1 will be treated every 3 days starting 2 weeks after banding, group 2 will be treated every 3 days starting 4 weeks after banding, group 4 will be treated every 3 days starting 6 weeks after banding. Mice will be killed at 8 weeks after banding. Treatment will be with rhBcl2-A1 or rhBim or saline. At the end of experiment, mice will have their cardiac function evaluated by ultra-sound, sacrifice by exsanguinations under anesthesia and tissue harvested for histology and biochemical assay. Tissue will be taken for evaluation of heart weight and lung weight and normalized to both body weight and dry weight.

Mouse Heart Failure—Ischemia

Mice will be pretreated with buprenorphine, anesthetized with isoflurane, their trachea intubated and attached to a ventilator. An incision will be made in the chest wall and a murine rib spreader inserted and the ribs gently spread to allow access to the thoracic cavity. The left cornary artery will be ligated at a position to result in 30-40% infarction of the left ventricle when measured at 4 weeks post ischemia. Infarcts of this size have been shown to result in heart failure. This procedure was reported to result in approximately 20% mortality in control mice as a result of ventricular rupture, an event that results in sudden death. Analgesics will be administered following the operation. Mice will be treated with various agents at times up to 6 days prior to ischemia or at times following the banding. Specifically, mice will be divided into treatment groups. Group 1 will be treated every 3 days starting 2 weeks after banding, group 2 will be treated every 3 days starting 4 weeks after banding, group 4 will be treated every 3 days starting 6 weeks after banding. Mice will be killed at 8 weeks after ischemia. Treatment will be with rhBcl2-A1 or Bim or saline. At the end of experiment, mice will have their cardiac function evaluated by ultra-sound, sacrifice by exsanguinations under anesthesia and tissue harvested for histology and biochemical assay. Tissue will be taken for evaluation of heart weight and lung weight and normalized to both body weight and dry weight.

We expect to find reduced lung weight to body weight and heart weight to body weight ration in all rhBcl2-A1 treated mice compared with the same measures in either rhBim or saline treated mice. In addition, we expect to find improved ejection fraction and reduced left ventricular diastolic diameter in all rhBcl2-A1 groups compared with rhBim or saline treated groups.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed is:

1. A method for inhibiting cell death or inflammation caused by heart failure in a mammal, the method comprising administering to a mammal in need thereof a Bcl protein in an amount sufficient to inhibit cell death and inflammation in the mammal.

2. The method of claim 1, wherein cell death is inhibited in the mammal.

3. The method of claim 1, wherein inflammation is inhibited in the mammal.

4. The method of claim 1, wherein cell death caused by acute heart failure is inhibited.

5. The method of claim 1, wherein the heart failure is chronic heart failure.

6. The method of claim 1, wherein cell death caused by chronic heart failure at a phase of acute exacerbation is inhibited.

7. The method of claim 1, wherein cell death caused by a phase of transition to chronic heart failure is inhibited.

8. The method of claim 1, wherein the administering is an acute administration regime.

9. The method of claim 1, wherein the administering is a chronic administration regime.

10. The method of claim 1, wherein the Bcl protein is administered intravenously.

11. The method of claim 1, wherein the Bcl protein is administered subcutaneously.

12. The method of claim 1, wherein the Bcl protein is administered orally.

13. The method of claim 1, wherein the Bcl protein is administered transdermally.

14. The method of claim 1, wherein the Bcl protein is a protein comprising at least 12 amino acids, wherein the protein is at least 50% similar to a segment of an A-1 protein, wherein the A-1 protein consists of the amino acid sequence set forth in SEQ ID NO:4;

15. The method of claim 1, wherein the Bcl protein is administered prophylactically to the mammal.

16. The method of claim 1, wherein the mammal is suffering heart failure.

17. The method of claim 1, wherein the Bcl protein is administered to the mammal in an amount from 0.5 μg/kg/day to 50 μg/kg/day for a period of time sufficient to inhibit a member of the group consisting of cell death and inflammation in the mammal.

18. The method of claim 14, wherein the Bcl protein is administered to the mammal for a period of from 1 day to 20 days.

19. The method of claim 14, wherein the Bcl protein is administered to the mammal on multiple occasions.

20. The method of claim 14, wherein the Bcl protein is administered to the mammal daily.
